SEQUENCE LISTING (20 sequences)

SEQ ID NO: 1 - protein, 667 aa, Synthetic construct
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGSGGSGGSGGSGGSGGSGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 2 - protein, 667 aa, Synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGSGGSGGSGGSGGSGGSGDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - protein, 827 aa, Synthetic construct
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSGGGGSDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGSGGSGGSGGSGGSGGSGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - protein, 827 aa, Synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGSGGSGGSGGSGGSGGSGDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSGGGGSDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 5 - protein, 145 aa, Homo sapiens
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 6 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 7 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 8 - protein, 18 aa, Synthetic construct
GSGGSGGSGGSGGSGGSG

SEQ ID NO: 9 - protein, 21 aa, Synthetic construct
GGGGSGGGGSGGGGSGGGGSG

SEQ ID NO: 10 - protein, 15 aa, Synthetic construct
GGGGSGGGGSGGGGS

SEQ ID NO: 11 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 12 - protein, 507 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 13 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 14 - protein, 347 aa, Synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 374 aa, Synthetic construct
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 12 aa, Homo sapiens
ESKYGPPCPPCP

SEQ ID NO: 17 - protein, 216 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 5 aa, Synthetic construct
GGGGS

SEQ ID NO: 19 - protein, 6 aa, Synthetic construct
GSGGSG

SEQ ID NO: 20 - protein, 386 aa, Synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

(19) United States
(12) Patent Application Publication
(10) Pub. No.: US 2024/0360219 A1
(43) Pub. Date: Oct. 31, 2024

(54) CD47/PD-L1-TARGETING PROTEIN COMPLEX AND METHODS OF USE THEREOF

(71) Applicant: FBD BIOLOGICS LIMITED, Kowloon (HK)

(72) Inventors: Jiin-Tarng Wang, Taichung City (TW); Chi-Ling Tseng, Taipei City (TW); Zong Sean Juo, New Taipei City (TW)

(21) Appl. No.: 18/769,347

(22) Filed: Jul. 10, 2024

Related U.S. Application Data
(63) Continuation of application No. PCT/US2022/053125, filed on Dec. 16, 2022.
(60) Provisional application No. 63/300,440, filed on Jan. 18, 2022.

(51) Int. Cl. C07K 16/28; A61P 35/00; C07K 16/22

(52) U.S. Cl. CPC C07K 16/2827; A61P 35/00; C07K 16/22; C07K 2317/31; C07K 2317/524; C07K 2317/526; C07K 2317/53

(57) ABSTRACT

This disclosure relates to protein complexes targeting CD47, PD-L1, and/or TGFβ, and methods of use thereof. In one aspect, the protein complexes include one or more CD47-binding domains, each including all or a portion of the SIRPα extracellular region; one or more PD-L1-binding domains, each including all or a portion of the PD-1 extracellular region; and optionally one or more TGFβ-binding domains, each including all or a portion of the TGFβR2 extracellular region.

### CROSS-REFERENCE TO RELATED APPLICATION

This application is a bypass continuation of and claims benefit under 35 U.S.C. § 120 to international Application No. PCT/US2022/053125 filed Dec. 16, 2022, which claims priority to and benefit of U.S. Provisional Patent Application Ser. No. 63/300,440, filed Jan. 18, 2022. Each of the foregoing applications is incorporated herein by reference in its entirety.

### SEQUENCE LISTING

This application contains a Sequence Listing that has been submitted electronically as an XML file named “52246-0002001.xml.” The XML file, created on Jul. 9, 2024, is 24,289 bytes in size. The material in the XML file is hereby incorporated by reference in its entirety.

### TECHNICAL FIELD

This disclosure relates to protein complexes targeting CD47 and PD-L1, and methods of use thereof.

### BACKGROUND

Signal regulatory protein α (SIRPα) is a regulatory membrane glycoprotein from SIRP family. It is mainly expressed by myeloid cells and also by stem cells or neurons. SIRPα acts as inhibitory receptor and interacts with a broadly expressed transmembrane protein CD47. This interaction negatively controls effector function of innate immune cells such as host cell phagocytosis. SIRPα diffuses laterally on the macrophage membrane and accumulates at a phagocytic synapse to bind CD47, which inhibits the cytoskeleton-intensive process of phagocytosis by the macrophage. CD47 provides a “do not eat” signal by binding to the N-terminus of signal regulatory protein alpha (SIRPα). CD47 has been found to be overexpressed in many different tumor cells. Targeting CD47 and/or SIRPα can be useful for cancer immunotherapy. However, because CD47 is also expressed on red blood cells (RBCs) and platelets, inhibiting the CD47/SIRPα interaction may cause phagocytosis of RBCs and platelets. Programmed death-1 (PD-1) is an immune checkpoint and guards against autoimmunity through a dual mechanism of promoting apoptosis in antigen-specific T-cells in lymph nodes while simultaneously reducing apoptosis in regulatory T cells. There is a need to develop therapies targeting CD47/SIRPα pathway and/or PD-1/PD-L1 pathway.

### SUMMARY

This disclosure relates to protein complexes targeting CD47 and PD-L1, and methods of use thereof. In some cases, the protein complex can also target TGFβ.

In one aspect, the disclosure is related to a protein complex, comprising: (a) an Fc; (b) a CD47-binding domain; and (c) a PD-L1 (programmed death-ligand 1)-binding domain. In some embodiments, the protein complex as described herein further comprises a TGFβ (transforming growth factor beta)-binding domain.

In some embodiments, the CD47-binding domain can bind to a cell (e.g., cancer cell) expressing CD47 and/or block the interaction between CD47 and signal regulatory protein α (SIRPα). In some embodiments, the CD47-binding domain is or comprises a SIRPα extracellular domain (e.g., a human SIRPα extracellular domain). In some embodiments, the CD47-binding domain is an anti-CD47 antibody or antigen-binding fragment thereof (e.g., a scFv or a VHH). In some embodiments, the CD47-binding domain comprises an amino acid sequence that is at least 80%, 85%, 90%, 95%, 96%, 97%, 98%, 99% or 100% identical to SEQ ID NO: 6.

In some embodiments, the PD-L1-binding domain can bind to a cell (e.g., cancer cell) expressing PD-L1 and/or block the interaction between PD-L1 and programmed cell death protein 1 (PD-1). In some embodiments, the PD-L1-binding domain is or comprises a PD-1 extracellular domain (e.g., a human PD-1 extracellular domain). In some embodiments, the PD-L1-binding domain is an anti-PD-L1 antibody or antigen-binding fragment thereof (e.g., a scFv or a VHH). In some embodiments, the PD-L1-binding domain comprises an amino acid sequence that is at least 80%, 85%, 90%, 95%, 96%, 97%, 98%, 99% or 100% identical to SEQ ID NO: 5.

In some embodiments, the TGFβ-binding domain can capture TGFβ thereby increasing immune response and/or improving tumor microenvironment. In some embodiments, the TGFβ-binding domain is or comprises a TGFBR2 extracellular domain (e.g., a human TGFBR2 extracellular domain). In some embodiments, the TGFβ-binding domain is an anti-TGFβ antibody or antigen-binding fragment thereof (e.g., a scFv or a VHH). In some embodiments, the TGFβ-binding domain comprises an amino acid sequence that is at least 80%, 85%, 90%, 95%, 96%, 97%, 98%, 99% or 100% identical to SEQ ID NO: 7.

In some embodiments, the Fc is human IgG4 Fc. In some embodiments, the CD47-binding domain is linked to the N-terminus of a CH2 domain in the Fc, optionally via a hinge region. In some embodiments, the PD-L1-binding domain is linked to the N-terminus of the CD47-binding domain, optionally via a linker peptide. In some embodiments, the PD-L1-binding domain is linked to the N-terminus of a CH2 domain in the Fc, optionally via a hinge region. In some embodiments, the CD47-binding domain is linked to the N-terminus of the PD-L1-binding domain, optionally via a linker peptide. In some embodiments, the hinge region is a human IgG4 hinge region optionally with S228P mutation according to EU numbering. In some embodiments, the TGFβ-binding domain is linked to the C-terminus of a CH3 domain in the Fc, optionally via a linker peptide.

In some embodiments, the protein complex comprises two or more CD47-binding domains. In some embodiments, the protein complex comprises two or more PD-L1-binding domains. In some embodiments, the protein complex comprises two or more TGFβ-binding domains.

In one aspect, the disclosure is related to a protein complex, comprising (a) a first polypeptide comprising from N-terminus to C-terminus: a first PD-L1-binding domain, an optional first linker peptide, a first CD47-binding domain, an optional first hinge region, a first Fc region, an optional second linker peptide, and an optional first TGFβ-binding domain; and (b) a second polypeptide comprising from N-terminus to C-terminus: a second PD-L1-binding domain, an optional third linker peptide, a second CD47-binding domain, an optional second hinge region, a second Fc region, an optional fourth linker peptide, and an optional second TGFβ-binding domain. In some embodiments, the first PD-L1-binding domain and/or the second PD-L1-binding domain comprise a sequence that is at least 80%, 90%, 95%, or 100% identical to SEQ ID NO: 5. In some embodiments, the first CD47-binding domain and/or the second CD47-binding domain comprise a sequence that is at least 80%, 90%, 95%, or 100% identical to SEQ ID NO: 6. In some embodiments, the first TGFβ-binding domain and/or the second TGFβ-binding domain comprise a sequence that is at least 80%, 90%, 95%, or 100% identical to SEQ ID NO: 7. In some embodiments, the first hinge region and/or the second hinge region comprise a sequence that is at least 80% identical to SEQ ID NO: 16. In some embodiments, the first Fc region and/or the second Fc region comprise a sequence that is at least 80% identical to SEQ ID NO: 17, optionally with a lysine or alanine residue added to the C-terminus. In some embodiments, the first linker peptide and/or the third linker peptide comprise a sequence that is at least 80% identical to SEQ ID NO: 8. In some embodiments, the second linker peptide and/or the fourth linker peptide comprise a sequence that is at least 80% identical to SEQ ID NO: 9. In some embodiments, the first polypeptide and/or the second polypeptide comprise a sequence that is at least 80%, 90%, 95%, or 100% identical to SEQ ID NO: 1.

In some embodiments, the first polypeptide further comprises a third PD-L1 binding domain, and the second polypeptide further comprises a fourth PD-L1-binding domain. In some embodiments, the third PD-L1-binding domain and/or the fourth PD-L1-binding domain comprise a sequence that is at least 80%, 90%, 95%, or 100% identical to SEQ ID NO: 5. In some embodiments, the third PD-L1-binding domain is linked to the N-terminus of the first PD-L1 binding domain, optionally via a fifth linker peptide, in some embodiments, the fourth PD-L1-binding domain is linked to the N-terminus of the second PD-L1-binding domain, optionally via a sixth linker peptide. In some embodiments, the fifth linker peptide and/or the sixth linker peptide comprise a sequence that is at least 80% identical to SEQ ID NO: 10. In some embodiments, the first polypeptide and/or the second polypeptide comprise a sequence that is at least 80%, 90%, 95%, or 100% identical to SEQ ID NO: 3.

In one aspect, the disclosure is related to a protein complex, comprising (a) a first polypeptide comprising from N-terminus to C-terminus: a first CD47-binding domain, an optional first linker peptide, a first PD-L1-binding domain, an optional first hinge region, a first Fc region, an optional second linker peptide, and an optional first TGFβ-binding domain; and (b) a second polypeptide comprising from N-terminus to C-terminus: a second CD47-binding domain, an optional third linker peptide, a second PD-L1-binding domain, an optional second hinge region, a second Fc region, an optional fourth linker peptide, and an optional second TGFβ-binding domain. In some embodiments, the first CD47-binding domain and/or the second CD47-binding domain comprise a sequence that is at least 80%, 90%, 95%, or 100% identical to SEQ ID NO: 6. In some embodiments, the first PD-L1-binding domain and/or the second PD-L1-binding domain comprise a sequence that is at least 80%, 90%, 95%, or 100% identical to SEQ ID NO: 5. In some embodiments, the first TGFβ-binding domain and/or the second TGFβ-binding domain comprise a sequence that is at least 80%, 90%, 95%, or 100% identical to SEQ ID NO: 7. In some embodiments, the first hinge region and/or the second hinge region comprise a sequence that is at least 80% identical to SEQ ID NO: 16. In some embodiments, the first Fc region and/or the second Fc region comprise a sequence that is at least 80% identical to SEQ ID NO: 17, optionally with a lysine or alanine residue added to the C-terminus. In some embodiments, the first linker peptide and/or the third linker peptide comprise a sequence that is at least 80% identical to SEQ ID NO: 8. In some embodiments, the second linker peptide and/or the fourth linker peptide comprise a sequence that is at least 80% identical to SEQ ID NO: 9. In some embodiments, the first polypeptide and/or the second polypeptide comprise a sequence that is at least 80%, 90%, 95%, or 100% identical to SEQ ID NO: 2.

In some embodiments, the first polypeptide further comprises a third PD-L1 binding domain, and the second polypeptide further comprises a fourth PD-L1-binding domain. In some embodiments, the third PD-L1-binding domain and/or the fourth PD-L1-binding domain comprise a sequence that is at least 80%, 90%, 95%, or 100% identical to SEQ ID NO: 5. In some embodiments, the third PD-L1-binding domain is fused between the first PD-L1-binding domain and the first hinge region, optionally via a fifth linker peptide between the first and the third PD-L1-binding domains, in some embodiments, the fourth PD-L1-binding domain is fused between the second PD-L1-binding domain and the second hinge region, optionally via a sixth linker peptide between the second and the fourth PD-L1-binding domains. In some embodiments, the fifth linker peptide and/or the sixth linker peptide comprise a sequence that is at least 80% identical to SEQ ID NO: 10. In some embodiments, the first polypeptide and/or the second polypeptide comprise a sequence that is at least 80%, 90%, 95%, or 100% identical to SEQ ID NO: 4.

In one aspect, the disclosure is related to a nucleic acid comprising a polynucleotide encoding the protein complex as described herein. In some embodiments, the nucleic acid is a DNA (e.g., cDNA) or RNA (e.g., mRNA).

In one aspect, the disclosure is related to a vector comprising one or more of the nucleic acids as described herein.

In one aspect, the disclosure is related to a cell comprising the vector as described herein. In some embodiments, the cell is a CHO cell.

In one aspect, the disclosure is related to a cell comprising one or more of the nucleic acids as described herein.

In one aspect, the disclosure is related to a method of producing a protein complex, the method comprising (a) culturing the cell as described herein under conditions sufficient for the cell to produce the protein complex; and (b) collecting the protein complex produced by the cell.

In one aspect, the disclosure is related to a protein conjugate comprising the protein complex as described herein, covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the protein complex as described herein, or the protein conjugate as described herein, to the subject. In some embodiments, the subject has a cancer cell expressing CD47 and/or PD-L1. In some embodiments, the cancer is breast cancer, prostate cancer, non-small cell lung cancer, pancreatic cancer, diffuse large B-cell lymphoma, mesothelioma, lung cancer, ovarian cancer, colon cancer, pleural tumor, glioblastoma, esophageal cancer, gastric cancer, synovial sarcoma, thymic carcinoma, endometrial carcinoma, stomach cancer, cholangiocarcinoma, head and neck cancer, blood cancer, or a combination thereof.

In one aspect, the disclosure is related to a method of decreasing the rate of tumor growth, the method comprising contacting a tumor cell with an effective amount of a composition comprising the protein complex as described herein, or the protein conjugate as described herein.

In one aspect, the disclosure is related to a method of killing a tumor cell, the method comprising contacting a tumor cell with an effective amount of a composition comprising the protein complex as described herein, or the protein conjugate as described herein.

In one aspect, the disclosure is related to a pharmaceutical composition comprising the protein complex as described herein, and a pharmaceutically acceptable carrier.

As used herein, the term “protein complex” or “protein construct” refers to a complex having one or more polypeptides. In some embodiments, the protein complex has two or more polypeptides, wherein the polypeptides can associate with each other, forming a dimer or a multimer.

As used herein, the term “CD47-binding domain” refers to a protein domain that can bind to CD47. In some embodiments, the CD47-binding domain can be an anti-CD47 antibody, an antigen-binding fragment thereof (e.g., a scFv or a VHH), or a CD47 binding protein or a portion thereof. In some embodiments, the CD47-binding domain can have one or more self-stabilizing domains. In some embodiments, the CD47-binding domain comprises or consists of a SIRPα extracellular domain. The SIRPα can be a wild type SIRPα, a human SIRPα, a polypeptide derived from a wildtype SIRPα (e.g., with mutations), or a portion thereof (e.g., the extracellular region of SIRPα, or IgV domain of SIRPα). In some embodiments, the polypeptide derived from a wildtype SIRPα can have one or more mutations. In some embodiments, the SIRPα extracellular domain comprises or consists of substantially the entire extracellular region of SIRPα or the variant thereof. In some embodiments, the SIRPα extracellular domain comprises or consists of the IgV domain of SIRPα or the variant thereof. In some embodiments, the IgV domain has one or more mutations. In some embodiments, the SIRPα extracellular domain has one or more mutations.

As used herein, the term “PD-L1-binding domain” refers to a protein domain that can bind to PD-L1. In some embodiments, the PD-L1-binding domain can be an anti-PD-L1 antibody, an antigen-binding fragment thereof (e.g., a scFv or a VHH), or a PD-L1-binding protein or a portion thereof. In some embodiments, the PD-L1-binding domain can have one or more self-stabilizing domains. In some embodiments, the PD-L1-binding domain comprises or consists of a PD-1 extracellular domain. The PD-1 can be a wild type PD-1, a human PD-1, a polypeptide derived from a wildtype PD-1 (e.g., with mutations), or a portion thereof (e.g., the extracellular region of PD-1). In some embodiments, the polypeptide derived from a wildtype PD-1 can have one or more mutations. In some embodiments, the PD-1 extracellular domain comprises or consists of substantially the entire extracellular region of PD-1 or the variant thereof. In some embodiments, the PD-1 extracellular domain comprises or consists of a portion of the extracellular region of PD-1 or the variant thereof. In some embodiments, the PD-1 extracellular domain has one or more mutations.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1D show schematic structures of HCB301-4, HCB301-3, HCB301-2, and HCB301-1, respectively.

FIG. 2 shows whole cell binding results of HCB301 proteins to transfected CHO—S cells expressing CD47.

FIG. 3 shows whole cell binding results of HCB301 proteins to transfected CHO—S cells expressing PD-L1.

FIG. 4 shows whole cell binding results of HCB301 proteins to CD47-expressing FaDu cells.

FIGS. 5A-5B show RBC binding results of HCB301 proteins. FIG. 5B shows results without the curve of Hu5F9-G4.

FIGS. 6A-6B show platelet binding results of HCB301 proteins. FIG. 6B shows results without the curve of Hu5F9-G4.

FIG. 7A shows human TGFβ1 binding results and EC50 values of HCB301 proteins. IgG4-TGFβ trap was used as a positive control.

FIG. 7B shows human TGFβ2 binding results and EC50 values of HCB301 proteins. IgG4-TGFβ trap was used as a positive control.

FIG. 7C shows human TGFβ3 binding results and EC50 values of HCB301 proteins. IgG4-TGFβ trap was used as a positive control.

FIG. 8A shows the binding percentage of HCB301 proteins at 32 pM concentration to untransfected OE19 cells and transfected OE19 cells expressing PD-L1.

FIGS. 8B-8G show the binding curves of SIRPα_G4, PD1_G4, HCB301-3, HCB301-4, HCB301-1, and HCB301-2 to untransfected OE19 cells and transfected OE19 cells expressing PD-L1.

FIG. 9 shows RBC hemagglutination results of HCB301 proteins.

FIG. 10 shows SIRPα/CD47 blocking results of HCB301 proteins on transfected CHO—S cells expressing CD47.

FIG. 11 shows PD-1/PD-L1 blocking results of HCB301 proteins on transfected CHO—S cells expressing PD-L1. Biotin-PD1-ECD-Fc stands for biotinylated PD1_G4.

FIG. 12A shows TGFβ-1-mediated smad2 reporter activity inhibited by HCB301 proteins.

FIG. 12B shows TGFβ-2-mediated smad2 reporter activity inhibited by HCB301 proteins.

FIG. 12C shows TGFβ-3-mediated smad2 reporter activity inhibited by HCB301 proteins.

FIG. 13 shows induced phagocytosis activity of HCB301 proteins by Raw264.7 mouse macrophages against CD47-expressing Jurkat cells.

FIG. 14A shows HCB301 protein-induced cell proliferation in MLR assays in the presence of TGFβ1.

FIG. 14B shows HCB301 protein-induced IL-2 secretion in MLR assays in the presence of TGFβ1 as determined by ELISA.

FIG. 14C shows HCB301 protein-induced IFN-γ secretion in MLR assays in the presence of TGFβ1 as determined by ELISA.

FIGS. 15A-15B show a table summarizing the in vitro assay results.

FIG. 16 lists sequences discussed in the disclosure.

### DETAILED DESCRIPTION

The present disclosure provides protein complexes binding to CD47 and PD-L1. These protein complexes can be used to target the CD47/SIRPα pathway and PD-1/PD-L1 pathway simultaneously. The results indicate that the protein complexes can effectively bind to CD47-expressing cancer cells and block the interaction between endogenous SIRPα and CD47, thereby inducing innate immune response (e.g., phagocytosis of cancer cells by macrophages). On the other hand, the protein complexes showed minimal binding to RBC cells or platelets, thereby inhibiting the clearance of host cells as observed by the anti-CD47 antibody magrolimab. In addition, the results indicate that the protein complexes can selectively bind to PD-L1-expressing cancer cells and block the interaction between endogenous PD-1 and PD-L1. Further, the protein complexes can also include a TGFβ trap, thereby inhibiting TGFβ-induced immune-suppressive response, increasing T cell proliferation and cytokine secretion.

Therefore, the protein complexes described herein can be used for cancer treatment with enhanced tumor immunogenicity and antigen presentation through increased phagocytosis by macrophages (e.g., by inactivation of CD47-mediated inhibition of phagocytosis); and enhanced T cell activation through inhibition of PD-1/PD-L1 as well as TGFβ signaling pathways.

### SIRPα Extracellular Domains

Signal regulatory protein α (SIRPα, SIRPa, Sirpa, or CD172A) is a transmembrane protein. It has an extracellular region comprising three Ig-like domains and a cytoplasmic region containing immunoreceptor tyrosine-based inhibition motifs that mediate binding of the protein tyrosine phosphatases SHP1 and SHP2. Tyrosine phosphorylation of SIRPα is regulated by various growth factors and cytokines as well as by integrin-mediated cell adhesion to extracellular matrix proteins. SIRPα is especially abundant in myeloid cells such as macrophages and dendritic cells, whereas it is expressed at only low levels in T, B, NK, and NKT cells.

The extracellular region of SIRPα can interact with its ligand CD47. The interaction of SIRPα on macrophages with CD47 on red blood cells prevents phagocytosis of Ig-opsonized red blood cells by macrophages in vitro and in vivo. The ligation of SIRPα on phagocytes by CD47 expressed on a neighboring cell results in phosphorylation of SIRPα cytoplasmic immunoreceptor tyrosine-based inhibition motifs, leading to the recruitment of SHP-1 and SHP-2 phosphatases. One resulting downstream effect is the prevention of myosin-IIA accumulation at the phagocytic synapse and consequently inhibition of phagocytosis. Thus, CD47-SIRPα interaction functions as a negative immune checkpoint to send a “don't eat me” signal to ensure that healthy autologous cells are not inappropriately phagocytosed. However, overexpression of CD47 has also been found in nearly all types of tumors, some of which include acute myeloid leukemia, non-Hodgkin's lymphoma, bladder cancer, and breast cancer. Such negative regulation of macrophages can be minimized by blocking the binding of CD47 to SIRPα. Thus, agents blocking CD47/SIRPα interaction can promote both antibody-dependent cellular phagocytosis (ADCP) and in some cases, trigger antibody-dependent cellular cytotoxicity (ADCC), thus can be used to treat various cancers.

Blocking CD47/SIRPα interaction can promote cellular phagocytosis, thus can be used to treat various cancers. It triggers the recognition and elimination of cancer cells by the innate immunity. Agents that target CD47 or SIRPα can be used to treat various tumors and cancers, e.g., solid tumors, hematologic malignancies (e.g., relapsed or refractory hematologic malignancies), acute myeloid leukemia, non-Hodgkin's lymphoma, breast cancer, bladder cancer, ovarian cancer, and small cell lung cancer tumors.

In addition, SIRPα acts to inhibit in vivo clearance of CD47-expressing host cells, including red blood cells and platelets, by macrophages. CD47-SIRPα interactions also appear essential for engraftment upon hematopoietic stem cells. Blocking CD47/SIRPα interaction may cause accidental killing of normal red blood cells, potentially resulting in anemia, and triggering inflammation. Thus, it is important to modulate the interaction of a SIRPα targeting agent with CD47, e.g., with limited or controlled effects on red blood cells.

A detailed description of SIRPα and its function can be found, e.g., in Yanagita et al. “Anti-SIRPα antibodies as a potential new tool for cancer immunotherapy.” JCI insight 2.1 (2017); Seiffert et al. “Signal-regulatory protein α (SIRPα) but not SIRPβ is involved in T-cell activation, binds to CD47 with high affinity, and is expressed on immature CD34+CD38-hematopoietic cells.” Blood 97.9 (2001): 2741-2749; which are incorporated by reference herein in the entirety.

Human SIRPα is a member of signal regulatory proteins (SIRPs). Signal regulatory proteins are cell surface Ig superfamily proteins that mediate essential cell surface protein interactions and signal transduction. SIRPs all contain an N-terminal extracellular region, a single transmembrane domain and a C-terminal intracellular region.

The extracellular region of human SIRPα (UniProt identifier: P78324) has an IgV domain, an Ig-like C1-type 1 domain, and an Ig-like C1-type 2 domain. They correspond to amino acids 32-137, amino acids 148-247, and amino acids 254-348 of the human SIRPα protein (SEQ ID NO: 23; NP_542970.1). Amino acids 1-30 are signal peptides. Human SIRPα also has a long intracellular domain that comprises two putative immunoreceptor tyrosine-based inhibition motifs (ITIM). Activation of SIRPα ITIMs delivers inhibitory signals that negatively regulate cell responses.

In some embodiments, the protein complex comprises one or more CD47-binding domains. In some embodiments, the CD47-binding domain comprises or consists of a SIRPα extracellular domain. As used herein, the “SIRPα extracellular domain” refers to the entire or a portion of the extracellular region of SIRPα or the variant thereof, wherein the portion of the extracellular region can bind to CD47. The SIRPα extracellular domain can have one or more protein domains that can fold independently and form self-stabilizing structures. In some embodiments, the SIRPα extracellular domain comprises or consists of one or more domains selected from an IgV domain, an Ig-like C1-type 1 domain, and an Ig-like C1-type 2 domain. In some embodiments, the SIRPα extracellular domain comprises or consists of an IgV domain. In some embodiments, the SIRPα extracellular domain comprises or consists of an IgV domain and an Ig-like C1-type 1 domain. In some embodiments, the SIRPα extracellular domain comprises or consists of an IgV domain, an Ig-like C1-type 1 domain, and an Ig-like C1-type 2 domain.

In some embodiments, the SIRPα extracellular domain described herein includes an amino acid sequence that is at least 80%, 85%, 90%, 95%, 96%, 97%, 98%, 99%, or 100% identical to amino acids 31-148 of human SIRPα protein (NCBI Accession No.: AAH26692.1; SEQ ID NO: 12). In some embodiments, the CD47-binding domain or SIRPα domain described herein includes an amino acid sequence that is at least 80%, 85%, 90%, 95%, 96%, 97%, 98%, 99%, or 100% identical to SEQ ID NO: 6. In some embodiments, the CD47-binding domain or SIRPα extracellular domain described herein includes the IgV domain of human SIRPα protein. In some embodiments, the CD47-binding domain or SIRPα extracellular domain described herein includes the IgV domain of mouse SIRPα protein.

### PD-1 Extracellular Domains

PD-1 (programmed death-1) is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 is mainly expressed on the surfaces of T cells and primary B cells; two ligands of PD-1 (PD-L1 and PD-L2) are widely expressed in antigen-presenting cells (APCs). The interaction of PD-1 with its ligands plays an important role in the negative regulation of the immune response. Inhibition the binding between PD-1 and its ligand can make the tumor cells exposed to the killing effect of the immune system, and thus can reach the effect of killing tumor tissues and treating cancers.

PD-L1 is expressed on the neoplastic cells of many different cancers. By binding to PD-1 on T-cells leading to its inhibition, PD-L1 expression is a major mechanism by which tumor cells can evade immune attack. PD-L1 over-expression may conceptually be due to 2 mechanisms, intrinsic and adaptive. Intrinsic expression of PD-L1 on cancer cells is related to cellular/genetic aberrations in these neoplastic cells. Activation of cellular signaling including the AKT and STAT pathways results in increased PD-L1 expression. In primary mediastinal B-cell lymphomas, gene fusion of the MHC class II transactivator (CIITA) with PD-L1 or PD-L2 occurs, resulting in over expression of these proteins. Amplification of chromosome 9p23-24, where PD-L1 and PD-L2 are located, leads to increased expression of both proteins in classical Hodgkin lymphoma. Adaptive mechanisms are related to induction of PD-L1 expression in the tumor microenvironment. PD-L1 can be induced on neoplastic cells in response to interferon γ. In microsatellite instability colon cancer, PD-L1 is mainly expressed on myeloid cells in the tumors, which then suppress cytotoxic T-cell function.

The use of PD-1 blockade to enhance anti-tumor immunity originated from observations in chronic infection models, where preventing PD-1 interactions reversed T-cell exhaustion. Similarly, blockade of PD-1 prevents T-cell PD-1/tumor cell PD-L1 or T-cell PD-1/tumor cell PD-L2 interaction, leading to restoration of T-cell mediated anti-tumor immunity.

A detailed description of PD-1, and the use of anti-PD-1 antibodies to treat cancers are described, e.g., in Topalian, Suzanne L., et al. “Safety, activity, and immune correlates of anti-PD-1 antibody in cancer.” New England Journal of Medicine 366.26 (2012): 2443-2454; Hirano, Fumiya, et al. “Blockade of B7-H1 and PD-1 by monoclonal antibodies potentiates cancer therapeutic immunity.” Cancer research 65.3 (2005): 1089-1096; Raedler, Lisa A. “Keytruda (pembrolizumab): first PD-1 inhibitor approved for previously treated unresectable or metastatic melanoma.” American health & drug benefits 8.Spec Feature (2015): 96; Kwok, Gerry, et al. “Pembrolizumab (Keytruda).” (2016): 2777-2789; US20170247454; U.S. Pat. Nos. 9,834,606 B; and 8,728,474; each of which is incorporated by reference in its entirety.

According to UniProt identifier Q15116, the extracellular region of human PD-1 corresponds to amino acids 24-170 of SEQ ID NO: 11, the transmembrane region of human PD-1 corresponds to amino acids 171-191 of SEQ ID NO: 11, and the cytoplasmic region of human PD-1 corresponds to amino acids 192-288 of SEQ ID NO: 11. The PD-1 extracellular region also has an IgV domain, which corresponds to amino acids 35-145 of the human PD-1 protein (SEQ ID NO: 11; NP_005009.2). The signal peptide corresponds to amino acids 1-23 of SEQ ID NO: 11. The cytoplasmic region of human SIRPα also has an immunoreceptor tyrosine-based inhibition motif (ITIM; corresponding to amino acids 221-226 of SEQ ID NO: 11) and a Immunoreceptor tyrosine-based switch motif (ITSM; corresponding to amino acids 241-251 of SEQ ID NO: 11).

In some embodiments, the protein complex comprises one or more PD-L1-binding domains. In some embodiments, the PD-L1-binding domain comprises or consists of a PD-1 extracellular domain. As used herein, the “PD-1 extracellular domain” refers to the entire or a portion of the extracellular region of PD-1 or the variant thereof, wherein the portion of the extracellular region can bind to PD-L1. The PD-1 extracellular domain can have one or more protein domains that can fold independently and form self-stabilizing structures. In some embodiments, the PD-1 extracellular domain comprises or consists of the IgV domain. In some embodiments, the PD-1 extracellular domain does not include the signal peptide.

In some embodiments, the PD-1 extracellular domain described herein includes an amino acid sequence that is at least 80%, 85%, 90%, 95%, 96%, 97%, 98%, 99%, or 100% identical to amino acids 26-170 of human PD-1 protein (NCBI Accession No.: NP_005009.2; SEQ ID NO: 11). In some embodiments, the PD-L1-binding domain or PD-1 extracellular domain described herein includes an amino acid sequence that is at least 80%, 85%, 90%, 95%, 96%, 97%, 98%, 99%, or 100% identical to SEQ ID NO: 5. In some embodiments, the PD-L1-binding domain or SIRPα extracellular domain described herein includes the IgV domain of human PD-1 protein. In some embodiments, the PD-L1-binding domain or SIRPα extracellular domain described herein includes all or a portion of the extracellular domain of mouse PD-L1 protein.

### TGFBR2 Extracellular Domains

TGF-beta receptor type-2 (TGFBR2) is the ligand-binding receptor for all members of the TGF-β family and expressed in virtually all cell types including fibroblasts. Ligand-induced cell response is mediated through either the canonical, Smad-dependent or non-canonical, Smad-independent signaling pathways such as c-Jun N-terminal kinase, Akt, Src, extracellular signal-regulated kinase and p38 mitogen-activated protein kinase pathway. Ligand binding to TGFBR2 leads to dimerization and autophosphorylation of the receptor, which then binds to TGF-beta receptor type-1 (TGFBR1) or type-3 (TGFBR3). The newly formed heterotetrameric complex in turn recruits and phosphorylates regulatory SMADs (SMAD2 or SMAD3), which in their phosphorylation state bind to co-SMAD molecule SMAD4. The regulatory SMAD/co-SMAD complex translocates to the nucleus where it acts as a transcription factor regulating target gene expression.

A detailed description of TGFβ, TGFBR2, and the use of TGFβ trap to treat cancers are described, e.g., in Bierie, B., et al. “TGFβ: the molecular Jekyll and Hyde of cancer.” Nature Reviews Cancer 6.7 (2006): 506-520; Kim, B., et al. “Novel therapies emerging in oncology to target the TGF-β pathway.” Journal of Hematology & Oncology 14.1 (2021): 1-20; and Lind, H., et al. “Dual targeting of TGF-β and PD-L1 via a bifunctional anti-PD-L1/TGF-βRII agent: status of preclinical and clinical advances.” Journal for immunotherapy of cancer 8.1 (2020); each of which is incorporated by reference in its entirety.

According to UniProt identifier P37173, the extracellular region of human TGFBR2 corresponds to amino acids 23-166 of SEQ ID NO: 13, the transmembrane region of human TGFBR2 corresponds to amino acids 167-187 of SEQ ID NO: 13, and the cytoplasmic region of human TGFBR2 corresponds to amino acids 188-567 of SEQ ID NO: 13. The signal peptide corresponds to amino acids 1-22 of SEQ ID NO: 13.

In some embodiments, the protein complex comprises one or more TGFβ-binding domains. In some embodiments, the TGFβ-binding domain comprises or consists of a TGFBR2 extracellular domain. As used herein, the “TGFBR2 extracellular domain” refers to the entire or a portion of the extracellular region of TGFBR2 or the variant thereof, wherein the portion of the extracellular region can bind to TGFβ. The TGFBR2 extracellular domain can have one or more protein domains that can fold independently and form self-stabilizing structures. In some embodiments, the TGFBR2 extracellular domain does not include the signal peptide.

In some embodiments, the TGFBR2 extracellular domain described herein includes an amino acid sequence that is at least 80%, 85%, 90%, 95%, 96%, 97%, 98%, 99%, or 100% identical to amino acids 24-159 of human TGFBR2 protein (NCBI Accession No.: NP_003233.4; SEQ ID NO: 13). In some embodiments, the TGFβ-binding domain or TGFBR2 extracellular domain described herein includes an amino acid sequence that is at least 80%, 85%, 90%, 95%, 96%, 97%, 98%, 99%, or 100% identical to SEQ ID NO: 7. In some embodiments, the TGFβ-binding domain or TGFBR2 extracellular domain described herein includes all or a portion of human TGFBR2 extracellular domain. In some embodiments, the TGFβ-binding domain or TGFBR2 extracellular domain described herein includes all or a portion of the extracellular domain of mouse PD-L1 protein.

Protein Complexes Targeting CD47, PD-L1, and/or TGFβ

The disclosure provides protein complexes that can specifically bind to CD47. In some embodiments, these protein complexes can block CD47/SIRPα signaling pathway thus increase immune response. In some embodiments, these protein complexes can initiate phagocytosis.

The disclosure also provides protein complexes that can specifically bind to PD-L1. In some embodiments, these protein complexes can block PD-1/PD-L1 signaling pathway thus increase immune response. In some embodiments, these protein complexes can induce T cell activation, proliferation, and/or cytokine release.

In one aspect, the disclosure provides a protein complex or a protein construct, comprising or consisting of an Fc, one or more CD47-binding domains, one or more PD-L1-binding domains, and optionally one or more TGFβ-binding domains. As used herein, the term “Fc” refers to the fragment crystallizable region of an antibody (e.g., IgG, IgE, IgM, IgA, or IgD). The term “Fc region” or “Fc region sequence” refers to heavy chain constant domains (e.g., CH2 and CH3) in a heavy chain peptide that form the Fc region. In some embodiments, the protein complex or the protein construct comprises 1, 2, 3, 4, 5, or 6 CD47-binding domains. In some embodiments, the protein complex or the protein construct comprises 1, 2, 3, 4, 5, or 6 PD-L1-binding domains. In some embodiments, the protein complex or the protein construct comprises 1, 2, 3, 4, 5, or 6 TGFβ-binding domains.

In some embodiments, the protein complex or the protein construct comprises or consists of an Fc, a first domain that specifically binds to cluster of differentiation 47 (CD47), and a second domain that specifically binds to programmed death-ligand 1 (PD-L1).

In some embodiments, the first domain can bind to a cell (e.g., cancer cell) expressing CD47 and/or block the interaction between CD47 and signal regulatory protein α (SIRPα). In some embodiments, the first domain comprises all or a portion of the extracellular region of SIRPα. In some embodiments, the SIRPα is human SIRPα. In some embodiments, the first domain comprises an amino acid sequence that is at least 80%, 85%, 90%, 95%, 96%, 97%, 98%, 99% or 100% identical to SEQ ID NO: 6.

In some embodiments, the second domain can bind to a cell (e.g., cancer cell) expressing PD-L1 and/or stimulate T cell activation and proliferation. In some embodiments, the second domain comprises all or a portion of the extracellular region of programmed cell death protein 1 (PD-1). In some embodiments, the PD-1 is human PD-1. In some embodiments, the second domain comprises an amino acid sequence that is at least 80%, 85%, 90%, 95%, 96%, 97%, 98%, 99% or 100% identical to SEQ ID NO: 5.

In some embodiments, the Fc is human IgG4 Fc. In some embodiments, the first domain is linked to the N-terminus of a CH2 domain in the Fc, optionally via a hinge region. In some embodiments, the second domain is linked to the N-terminus of a CH2 domain in the Fc, optionally via a hinge region. In some embodiments, the hinge region is a human IgG4 hinge region optionally with S228P mutation according to EU numbering.

In some embodiments, the protein complex or the protein construct further comprises a third domain that specifically binds to transforming growth factor beta (TGFβ). In some embodiments, the third domain is linked to the C-terminus of a CH3 domain in the Fc, optionally via a linker peptide.

In some embodiments, the protein complex comprises two or more first domains. In some embodiments, the protein complex comprises two or more second domains. In some embodiments, the protein complex comprises two or more third domains.

In some embodiments, the CD47-binding domains, the PD-L1-binding domains, and the TGFβ-binding domains are linked to the Fc region through any of the linker peptide or the hinge region sequence as described herein.

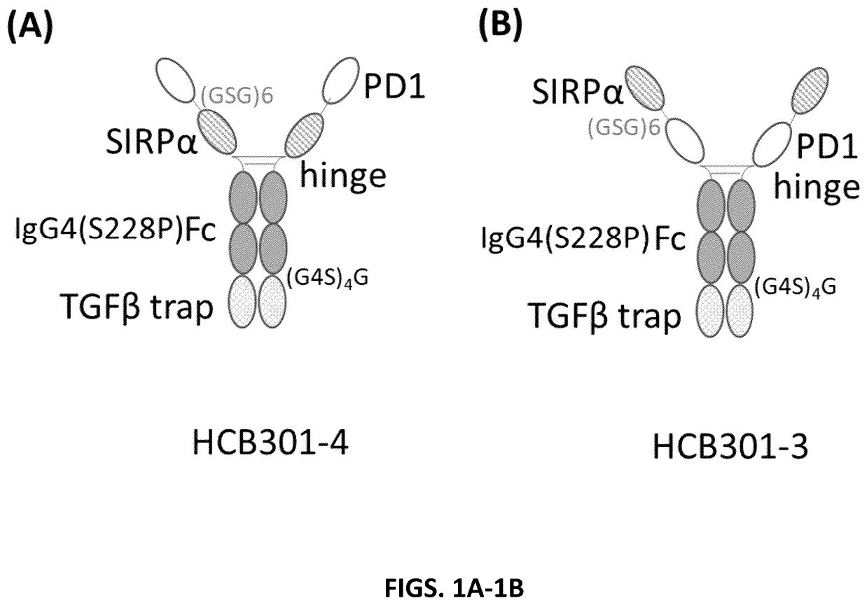

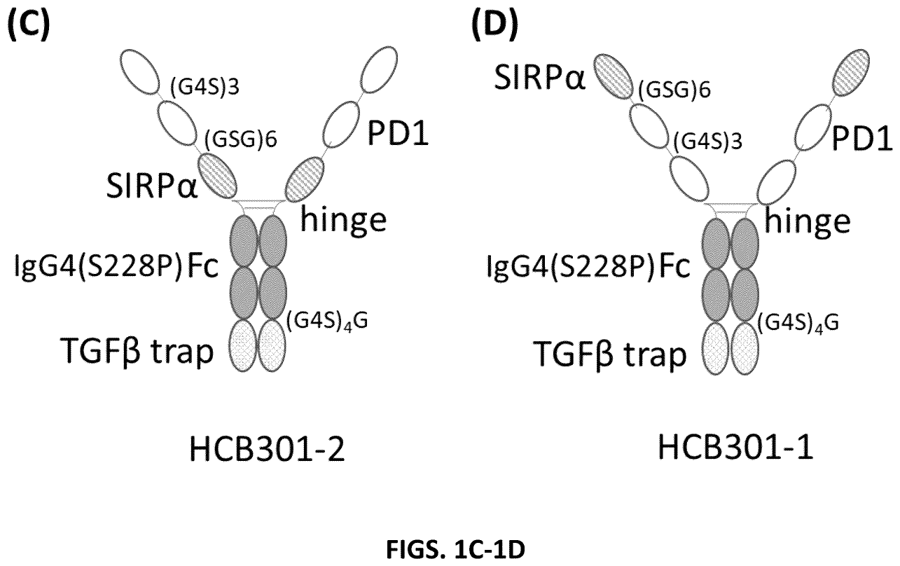

Some embodiments of the protein complexes are shown in FIGS. 1A-1D. They are described in detail below.

### HCB301-4

In one aspect, the disclosure is related to a protein complex including a first polypeptide and a second polypeptide. The first polypeptide includes, preferably from N-terminus to C-terminus, a first PD-L1-binding domain, an optional first linker peptide, a first CD47-binding domain, an optional first hinge region, a first Fc region, an optionally a second linker peptide, and an optional first TGFβ-binding domain. The second polypeptide includes, preferably from N-terminus to C-terminus, a second PD-L1-binding domain, an optional third linker peptide, a second CD47-binding domain, an optional second hinge region, a second Fc region, an optional fourth linker peptide, and an optional second TGFβ-binding domain. A schematic structure of an exemplary protein complex having a HCB301-4 format is shown in FIG. 1A.

In some embodiments, the first and/or the second PD-L1-binding domains include all or a portion of the extracellular domain of PD-1, e.g., amino acids 26-170 of human PD-1 protein (NCBI Accession No.: NP_005009.2; SEQ ID NO: 11); or SEQ ID NO: 5. In some embodiments, the first and/or the second PD-L1-binding domains are identical. In some embodiments, the first and/or the second PD-L1-binding domains are different. In some embodiments, the first and/or the second PD-L1-binding domains include an amino acid sequence that is at least 80%, 85%, 90%, 91%, 92%, 93%, 94%, 95%, 96%, 97%, 98%, 99%, or 100% identical to SEQ ID NO: 5.

In some embodiments, the first, the second, the third, and/or the fourth linker peptide described herein include an amino acid sequence that is at least 80%, 85%, 90%, 95%, or 100% identical to any one of SEQ ID NOs: 8-10. In some embodiments, the first, the second, the third, and/or the fourth linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 18) or GSGGSG (SEQ ID NO: 19).

In some embodiments, the first and/or the second polypeptide include an amino acid sequence that is at least 80%, 85%, 90%, 91%, 92%, 93%, 94%, 95%, 96%, 97%, 98%, 99%, or 100% identical to SEQ ID NO: 1.

### HCB301-2

In one aspect, the disclosure is related to a protein complex including a first polypeptide and a second polypeptide. The first polypeptide includes, preferably from N-terminus to C-terminus, a third PD-L1-binding domain, an optional fifth linker peptide, a first PD-L1-binding domain, an optionally first linker peptide, a first CD47-binding domain, an optional first hinge region, a first Fc region, an optionally a second linker peptide, and an optional first TGFβ-binding domain. The second polypeptide includes, preferably from N-terminus to C-terminus, a fourth PD-L1-binding domain, an optionally sixth linker peptide, a second PD-L1-binding domain, an optional third linker peptide, a second CD47-binding domain, an optional second hinge region, a second Fc region, an optional fourth linker peptide, and an optional second TGFβ-binding domain. A schematic structure of an exemplary protein complex having a HCB301-2 format is shown in FIG. 1C.

In some embodiments, the first, the second, the third, and/or the fourth PD-L1-binding domains include all or a portion of the extracellular domain of PD-1, e.g., amino acids 26-170 of human PD-1 protein (NCBI Accession No.: NP_005009.2; SEQ ID NO: 11); or SEQ ID NO: 5. In some embodiments, the first, the second, the third, and/or the fourth PD-L1-binding domains are identical. In some embodiments, the first, the second, the third, and/or the fourth PD-L1-binding domains are different. In some embodiments, the first, the second, the third, and/or the fourth PD-L1-binding domains include an amino acid sequence that is at least 80%, 85%, 90%, 91%, 92%, 93%, 94%, 95%, 96%, 97%, 98%, 99%, or 100% identical to SEQ ID NO: 5.

In some embodiments, the first, the second, the third, the fourth, the fifth, and/or the sixth linker peptide described herein include an amino acid sequence that is at least 80%, 85%, 90%, 95%, or 100% identical to any one of SEQ ID NOs: 8-10. In some embodiments, the first, the second, the third, the fourth, the fifth, and/or the sixth linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 18) or GSGGSG (SEQ ID NO: 19).

In some embodiments, the first and/or the second polypeptide include an amino acid sequence that is at least 80%, 85%, 90%, 91%, 92%, 93%, 94%, 95%, 96%, 97%, 98%, 99%, or 100% identical to SEQ ID NO: 3.

### HCB301-3

In one aspect, the disclosure is related to a protein complex including a first polypeptide and a second polypeptide. The first polypeptide includes, preferably from N-terminus to C-terminus, a first CD47-binding domain, an optional first linker peptide, a first PD-L1-binding domain, an optional first hinge region, a first Fc region, an optionally a second linker peptide, and an optional first TGFβ-binding domain. The second polypeptide includes, preferably from N-terminus to C-terminus, a second CD47-binding domain, an optional third linker peptide, a second PD-L1-binding domain, an optional second hinge region, a second Fc region, an optional fourth linker peptide, and an optional second TGFβ-binding domain. A schematic structure of an exemplary protein complex having a HCB301-4 format is shown in FIG. 1B.

In some embodiments, the first and/or the second PD-L1-binding domains include all or a portion of the extracellular domain of PD-1, e.g., amino acids 26-170 of human PD-1 protein (NCBI Accession No.: NP_005009.2; SEQ ID NO: 11); or SEQ ID NO: 5. In some embodiments, the first and/or the second PD-L1-binding domains are identical. In some embodiments, the first and/or the second PD-L1-binding domains are different. In some embodiments, the first and/or the second PD-L1-binding domains include an amino acid sequence that is at least 80%, 85%, 90%, 91%, 92%, 93%, 94%, 95%, 96%, 97%, 98%, 99%, or 100% identical to SEQ ID NO: 5.

In some embodiments, the first, the second, the third, and/or the fourth linker peptide described herein include an amino acid sequence that is at least 80%, 85%, 90%, 95%, or 100% identical to any one of SEQ ID NOs: 8-10. In some embodiments, the first, the second, the third, and/or the fourth linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 18) or GSGGSG (SEQ ID NO: 19).

In some embodiments, the first and/or the second polypeptide include an amino acid sequence that is at least 80%, 85%, 90%, 91%, 92%, 93%, 94%, 95%, 96%, 97%, 98%, 99%, or 100% identical to SEQ ID NO: 2.

### HCB301-1

In one aspect, the disclosure is related to a protein complex including a first polypeptide and a second polypeptide. The first polypeptide includes, preferably from N-terminus to C-terminus, a first CD47-binding domain, an optionally first linker peptide, a first PD-L1-binding domain, an optional fifth linker peptide, a third PD-L1-binding domain, an optional first hinge region, a first Fc region, an optionally a second linker peptide, and an optional first TGFβ-binding domain. The second polypeptide includes, preferably from N-terminus to C-terminus, a second CD47-binding domain, an optional third linker peptide, a second PD-L1-binding domain, an optionally sixth linker peptide, a fourth PD-L1-binding domain, an optional second hinge region, a second Fc region, an optional fourth linker peptide, and an optional second TGFβ-binding domain. A schematic structure of an exemplary protein complex having a HCB301-2 format is shown in FIG. 1D.

In some embodiments, the first, the second, the third, and/or the fourth PD-L1-binding domains include all or a portion of the extracellular domain of PD-1, e.g., amino acids 26-170 of human PD-1 protein (NCBI Accession No.: NP_005009.2; SEQ ID NO: 11); or SEQ ID NO: 5. In some embodiments, the first, the second, the third, and/or the fourth PD-L1-binding domains are identical. In some embodiments, the first, the second, the third, and/or the fourth PD-L1-binding domains are different. In some embodiments, the first, the second, the third, and/or the fourth PD-L1-binding domains include an amino acid sequence that is at least 80%, 85%, 90%, 91%, 92%, 93%, 94%, 95%, 96%, 97%, 98%, 99%, or 100% identical to SEQ ID NO: 5.

In some embodiments, the first, the second, the third, the fourth, the fifth, and/or the sixth linker peptide described herein include an amino acid sequence that is at least 80%, 85%, 90%, 95%, or 100% identical to any one of SEQ ID NOs: 8-10. In some embodiments, the first, the second, the third, the fourth, the fifth, and/or the sixth linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 18) or GSGGSG (SEQ ID NO: 19).

In some embodiments, the first and/or the second polypeptide include an amino acid sequence that is at least 80%, 85%, 90%, 91%, 92%, 93%, 94%, 95%, 96%, 97%, 98%, 99%, or 100% identical to SEQ ID NO: 4.

In any of the protein complexes described herein, the first and/or the second CD47-binding domains can include all or a portion of the extracellular domain of SIRPα, e.g., amino acids 31-148 of human SIRPα protein (NCBI Accession No.: AAH26692.1; SEQ ID NO: 12); or SEQ ID NO: 6. In some embodiments, the first and the second CD47-binding domains are identical. In some embodiments, the first and/or the second CD47-binding domain include an amino acid sequence that is at least 80%, 85%, 90%, 91%, 92%, 93%, 94%, 95%, 96%, 97%, 98%, 99%, or 100% identical to SEQ ID NO: 6. In some embodiments, the first and/or the second CD47-binding domains include the IgV domain of SIRPα (e.g., human SIRPα). In some embodiments, the SIRPα IgV domain includes one or more mutations. In some embodiments, the first and the second CD47-binding domains are different.

In any of the protein complexes described herein, the first and/or the second TGFβ-binding domains can include all or a portion of the extracellular domain of TGFBR2, e.g., amino acids 24-159 of human TGFBR2 protein (NCBI Accession No.: NP_003233.4; SEQ ID NO: 13); or SEQ ID NO: 7. In some embodiments, the first and/or the second TGFβ-binding domains are identical. In some embodiments, the first and/or the second TGFβ-binding domains are different. In some embodiments, the first and/or the second TGFβ-binding domains include an amino acid sequence that is at least 80%, 85%, 90%, 91%, 92%, 93%, 94%, 95%, 96%, 97%, 98%, 99%, or 100% identical to SEQ ID NO: 7.

In any of the protein complexes described herein, the first and/or the second hinge region can include all or a portion of the hinge region of an immunoglobulin, e.g., human IgG4 hinge region (SEQ ID NO: 16). In some embodiments, the first and/or the second hinge region include an amino acid sequence that is at least 80%, 85%, 90%, 91%, 92%, 93%, 94%, 95%, 96%, 97%, 98%, 99%, or 100% identical to SEQ ID NO: 16. In some embodiments, the first and the second hinge regions are identical. In some embodiments, the first and the second hinge regions are different. In some embodiments, the first and/or the second hinge region include a proline at position 228 according to EU numbering.

In any of the protein complexes described herein, the first and/or the second Fc region can be identical and can form a Fc homodimer. In some embodiments, the first and/or the second Fc region include all or a portion of the Fc region of an immunoglobulin, e.g., human IgG4 Fc region (SEQ ID NO: 17). In any of the protein complexes described herein, the first and/or the second Fc region can be different. In some embodiments, the first and/or the second Fc region can form a Fc heterodimer by introducing one or more mutations. For example, the first and/or the second Fc region can include one or more knob-into-hole (KIH) mutations. In some embodiments, the first and/or the second Fc region can form a Fc heterodimer using other technologies known in the art. Details heterodimeric Fc technologies can be found, e.g., in Ha, et al. “Immunoglobulin Fc heterodimer platform technology: from design to applications in therapeutic antibodies and proteins.” Frontiers In Immunology 7 (2016): 394, which is incorporated herein by reference in its entirety.

In one aspect, the disclosure is related to a protein complex including a CD47-binding domain (e.g., any of the CD47-binding domain described herein) and a PD-L1-binding domain (e.g., any of the PD-L1-binding domain described herein). In some embodiments, the protein complex further include a TGFβ-binding domain (e.g., any of the TGFβ-binding domain described herein). In some embodiments, the CD47-binding domain is or comprises a SIRPα extracellular domain (e.g., any of the SIRPα extracellular domain described herein). In some embodiments, the PD-L1-binding domain is or comprises a PD-1 extracellular domain (e.g., any of the PD-1 extracellular domain described herein). In some embodiments, the TGFβ-binding domain is or comprises a TGFBR2 extracellular domain (e.g., any of the TGFBR2 extracellular domain described herein).

### Characteristics of Protein Complexes

In some embodiments, the protein complex can comprise any CD47-binding domains, PD-L1-binding domains, and/or TGFβ-binding domains as described herein. The disclosure also provides nucleic acid comprising a polynucleotide encoding a polypeptide described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The protein complex described herein can include an Fc of an antibody. These antibodies can be of any type (e.g., IgG, IgE, IgM, IgD, IgA, and IgY), class or subclass (e.g., IgG1, IgG2, IgG3, IgG4, IgA1, IgA2, IgE1, IgE2). In some embodiments, the Fc region is derived from human IgG (e.g., IgG1, IgG2, IgG3, or IgG4). In some embodiments, the Fc region is an IgG4 Fc region (e.g., human IgG4 Fc region).

In some embodiments, the protein complex described herein is linked to the Fc region through an antibody hinge region (e.g., IgG, IgE hinge region). In addition, the Fc region can be modified to provide desired effector functions or serum half-life.

The protein complex described herein can block the binding between CD47 and endogenous SIRPα that are expressed on immune cells. In some embodiments, by binding to CD47, the protein complex described herein can inhibit the binding of CD47 (e.g., that is expressed on tumor cells) to endogenous SIRPα that is expressed on immune cells (e.g., myeloid cells, macrophages and dendritic cells), thereby blocking CD47/SIRPα pathway, upregulating immune response, and promoting phagocytosis.

The protein complex described herein can block the binding between PD-L1 and endogenous PD-1 that are expressed on immune cells. In some embodiments, by binding to PD-L1, the protein complex described herein can inhibit the binding of PD-L1 (e.g., that is expressed on tumor cells) to endogenous PD-1 that is expressed on immune cells (e.g., T cells), thereby blocking PD-1/PD-L1 pathway, upregulating immune response, activating T cell proliferation and cytokine release.

In some embodiments, the protein complex described herein can increase immune response, activity or number of immune cells (e.g., myeloid cells, macrophages, dendritic cells, antigen presenting cells) by at least 10%, 20%, 30%, 40%, 50%, 60%, 70%, 80%, 90%, 100%, 2 folds, 3 folds, 5 folds, 10 folds, or 20 folds.

In some implementations, the protein complex described herein can bind to CD47 (e.g., human CD47, monkey CD47, or mouse CD47), PD-L1 (e.g., human PD-L1, monkey PD-L1, or mouse PD-L1), or TGFβ (e.g., human TGFβ, monkey TGFβ, or mouse TGFβ) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms. Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, or 10 pM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity include, e.g., ELISA, RIA, and surface plasmon resonance (SPR). In some embodiments, the protein complex described herein can bind to monkey CD47, and/or mouse CD47. In some embodiments, the protein complex described herein cannot bind to monkey CD47, and/or mouse CD47. In some embodiments, the protein complex described herein can bind to monkey PD-L1, and/or mouse PD-L1. In some embodiments, the protein complex described herein cannot bind to monkey PD-L1, and/or mouse PD-L1. In some embodiments, the protein complex described herein can bind to monkey TGFβ, and/or mouse TGFβ. In some embodiments, the protein complex described herein cannot bind to monkey TGFβ, and/or mouse TGFβ.

In some embodiments, thermal stabilities are determined. The protein complex described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the protein complex described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the protein complex described herein has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the tumor inhibitory effects of the protein complex described herein are comparable to an anti-CD47 reference antibody, e.g., Hu5F9-G4, or an anti-SIRPα antibody, e.g., CC-95251. Hu5F9-G4 is described e.g., in Sikic et al. “First-in-human, first-in-class phase I trial of the anti-CD47 antibody Hu5F9-G4 in patients with advanced cancers.” Journal of Clinical Oncology 37.12 (2019): 946, which is incorporated herein by reference in its entirety. In some embodiments, the tumor inhibitory effects of the protein complex described herein are at least 10%, 20%, 30%, 40%, 50%, 60%, 70%, 80%, 90%, 1 fold, 2 folds, or 5 folds more than an anti-CD47 reference antibody, e.g., Hu5F9-G4, or an anti-SIRPα antibody, e.g., CC-95251. In some embodiments, the tumor inhibitory effects of the protein complex described herein are at least 10%, 20%, 30%, 40%, 50%, 60%, 70%, 80%, 90%, 1 fold, 2 folds, or 5 folds more than SIRPα_G4. Details of SIRPα_G4 (or hSIRPα-G4Fc-wt (Trillium), TTI-622) can be found, e.g., in U.S. Patent Application Publication No. US20150329616A1, which is incorporated herein by reference in its entirety. Amino acid sequence of SIRPα_G4 is shown in SEQ ID NO: 14.

In some embodiments, the tumor inhibitory effects of the protein complex described herein are comparable to an anti-PD-L1 reference antibody, e.g., MPDL3280A (atezolizumab), or an anti-PD-1 antibody, e.g., pembrolizumab. MPDL3280A is described e.g., in Powles, T. et al. “MPDL3280A (anti-PD-L1) treatment leads to clinical activity in metastatic bladder cancer.” Nature 515.7528 (2014): 558-562, which is incorporated herein by reference in its entirety. In some embodiments, the tumor inhibitory effects of the protein complex described herein are at least 10%, 20%, 30%, 40%, 50%, 60%, 70%, 80%, 90%, 1 fold, 2 folds, or 5 folds more than an anti-PD-L1 reference antibody, e.g., MPDL3280A, or an anti-PD-1 antibody, e.g., pembrolizumab. In some embodiments, the tumor inhibitory effects of the protein complex described herein are at least 10%, 20%, 30%, 40%, 50%, 60%, 70%, 80%, 90%, 1 fold, 2 folds, or 5 folds more than PD1_G4. PD1_G4 is used as a control protein for the HCB301 fusion proteins (HCB301 proteins). Amino acid sequence of PD1_G4 is shown in SEQ ID NO: 15.

In some embodiments, the tumor inhibitory effects of the protein complex described herein are comparable to an TGFβ trap protein, e.g., IgG4-TGFβ trap, or an anti-PD-L1 antibody×TGFβ trap M7824. M7824 is described e.g., in Gatti-Mays, M. E., et al. “M7824: a promising new strategy to combat cancer immune evasion.” Oncoscience 5.11-12 (2018): 269, which is incorporated herein by reference in its entirety. In some embodiments, the tumor inhibitory effects of the protein complex described herein are at least 10%, 20%, 30%, 40%, 50%, 60%, 70%, 80%, 90%, 1 fold, 2 folds, or 5 folds more than IgG4-TGFβ trap or M7824. In some embodiments, the tumor inhibitory effects of the protein complex described herein are at least 10%, 20%, 30%, 40%, 50%, 60%, 70%, 80%, 90%, 1 fold, 2 folds, or 5 folds more than IgG4-TGFβ trap. IgG4-TGFβ trap is used as a control protein for the HCB301 fusion proteins (HCB301 proteins). Amino acid sequence of IgG4-TGFβ trap is shown in SEQ ID NO: 20.

In some embodiments, the protein complex described herein has a functional Fc. In some embodiments, the Fc is from human IgG1, human IgG2, human IgG3, or human IgG4. In some embodiments, effector function of a functional Fc is antibody-dependent cell-mediated cytotoxicity (ADCC). In some embodiments, effector function of a functional Fc is phagocytosis. In some embodiments, effector function of a functional Fc is ADCC and phagocytosis. In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the main peak of HPLC-SEC accounts for at least 80%, 85%, 90%, 95%, 96%, 97%, 98%, 99%, or 99.5% of the protein complex described herein after purification by protein A-based affinity chromatography and/or size-exclusive chromatography.

In some embodiments, the protein complex described herein can bind to human CD47-expressing tumor cells (e.g., human CD47 tf CHO—S cells, or FaDu cells) with an affinity that is at least 10%, at least 20%, 30%, at least 40%, at least 50%, at least 60%, at least 70%, at least 80%, at least 90%, at least 100%, at least 110%, or at least 120% as compared to that an anti-CD47 reference antibody (e.g., Hu5F9-G4) or SIRPα_G4.

In some embodiments, the protein complex described herein can bind to human PD-L1-expressing tumor cells (e.g., transfected CHO—S cells expressing human PD-L1) with an affinity that is at least 10%, at least 20%, 30%, at least 40%, at least 50%, at least 60%, at least 70%, at least 80%, at least 90%, at least 100%, at least 110%, or at least 120% as compared to that of PD1_G4.

In some embodiments, the protein complex described herein can bind to human TGFβ (e.g., TGFβ1, TGFβ2, or TGFβ3) with an affinity that is at least 30%, at least 40%, at least 50%, at least 60%, at least 70%, at least 80%, at least 90%, at least 100%, at least 110%, or at least 120% as compared to that of IgG4-TGFβ trap. In some embodiments, the EC50 value of the protein complex binding to human TGFβ (e.g., TGFβ1, TGFβ2, or TGFβ3) is less than 10%, less than 20%, less than 30%, less than 40%, less than 50%, less than 60%, less than 70%, less than 80%, less than 90%, less than 1-fold, less than 2-fold, less than 3-fold, less than 4-fold, less than 5-fold, or less than 10-fold as compared to that of IgG4-TGFβ trap.

In some embodiments, the protein complex described herein can bind to RBC cells or platelets (e.g., from human donors) with an affinity that is less than 90%, less than 80%, less than 70%, less than 60%, less than 50%, less than 40%, less than 30%, less than 20%, less than 10%, less than 5%, less than 3%, or less than 1% as compared to that of an anti-CD47 reference antibody (e.g., Hu5F9-G4).

In some embodiments, the protein complex described herein does not induce hemagglutination. In some embodiments, the protein complex described herein can induce hemagglutination at a minimal concentration that is greater than 500-fold, 2000-fold, 5000-fold, 20000-fold, or 50000-fold as compared to that of an anti-CD47 reference antibody (e.g., Hu5F9-G4).

In some embodiments, the protein complex described herein can block the interaction between CD47 (e.g., human CD47 or fragments thereof) and SIRPα (e.g., human SIRPα or fragments thereof). In some embodiments, the protein complex described herein can block the interaction between human CD47-expressing cells (e.g., CD47 tf CHO—S cells or FaDu cells) and human SIRPα. In some embodiments, the blocking ability of the protein complex described herein is at least 30%, at least 50%, at least 60%, at least 70%, at least 80%, at least 90%, at least 100%, at least 110%, at least 120%, at least 130%, at least 140%, or at least 150% as compared to that an anti-CD47 reference antibody (e.g., Hu5F9-G4) or SIRPα_G4.

In some embodiments, the protein complex described herein can block the interaction between PD-L1 (e.g., human PD-L1 or fragments thereof) and PD-1 (e.g., human PD-1 or fragments thereof). In some embodiments, the protein complex described herein can block the interaction between human PD-L1-expressing cells (e.g., PD-L1 tf CHO—S cells) and human PD-1. In some embodiments, the blocking ability of the protein complex described herein is at least 30%, at least 50%, at least 60%, at least 70%, at least 80%, at least 90%, at least 100%, at least 110%, at least 120%, at least 130%, at least 140%, or at least 150% as compared to that an anti-PD-L1 reference antibody (e.g., MPDL3280A) or PD1_G4.

In some embodiments, the protein complex described herein can induce phagocytosis of CD47-expressing tumor cells (e.g., Jurkat cells) by mouse macrophages (e.g., Raw264.7 cells). In some embodiments, the EC50 value of the protein complex described herein to induce phagocytosis of CD47-expressing tumor cells is less than 30 nM, less than 20 nM, less than 10 nM, less than 5 nM, less than 4 nM, less than 3 nM, less than 2 nM, or less than 1 nM. In some embodiments, the EC50 value of the protein complex described herein to induce phagocytosis of CD47-expressing tumor cells is comparable to that of an anti-CD47 reference antibody (e.g., Hu5F9-G4) or SIRPα_G4. In some embodiments, the ability of the protein complex described herein to induce phagocytosis of CD47-expressing tumor cells by mouse microphages is at least 10%, at least 20%, at least 30%, at least 40%, at least 50%, at least 60%, at least 70%, at least 80%, or at least 90% as compared to that of Hu5F9-G4 or SIRPα_G4. In some embodiments, the protein complex described herein has a weaker ability (e.g., less than 80%, 70%, 60%, 50%, 40%, 30%, 20% or 10%) to induce phagocytosis of RBC cells or platelets by mouse macrophages (e.g., Raw264.7 cells) than an anti-CD47 reference antibody (e.g., Hu5F9-G4).

In some embodiments, the protein complex described herein can induce phagocytosis of CD47-expressing tumor cells (e.g., Jurkat cells) by human macrophages (e.g., MDM cells). In some embodiments, the ability of the protein complex described herein to induce phagocytosis of CD47-expressing tumor cells by human macrophages is at least 10%, at least 20%, at least 30%, at least 40%, at least 50%, at least 60%, at least 70%, at least 80%, or at least 90% as compared to that of an anti-CD47 reference antibody (e.g., Hu5F9-G4) or SIRPα G4. In some embodiments, the protein complex described herein has a weaker ability (less than 80%, 70%, 60%, 50%, 40%, 30%, 20% or 10%) to induce phagocytosis of RBC cells or platelets by human macrophages (e.g., MDM cells) than an anti-CD47 reference antibody (e.g., Hu5F9-G4).

Endogenous expression of CD47 on a variety of cell types, including red blood cells, creates a formidable “antigen sink” that may limit the efficacy of CD47-targeting therapies. Thus, the weaker ability of the protein complex described herein to induce phagocytosis of RBC cells and/or platelets may increase the in vivo efficacy of the protein complex. In addition, the protein complex may be administered with a lower dose level and/or less frequent dosage schedule with similar efficacy than an anti-CD47 reference antibody (e.g., Hu5F9-G4).

In some embodiments, the protein complex described herein can inhibit TGFβ-induced downstream pathways, e.g., smad2 reporter pathway. In some embodiments, the protein complex can inhibit TGFβ1-mediated smad2 reporter activity to less than 150%, less than 140%, less than 130%, less than 120%, less than 110%, less than 100%, less than 90%, less than 80%, less than 70%, less than 60%, or less than 50% as compared to that of IgG4-TGFβ trap, anti-TGFβ, or M7824.

In some embodiments, the protein complex described herein can enhance T cell response (e.g., in an MLR assay). The principle of a mixed lymphocyte reaction (MLR) is that T cells from one donor will proliferate in the presence of APCs from a different donor. This is caused by the recognition of an HLA mismatch between two unrelated donors, which provokes an immune response from the T cells. MLR is often used as a means of inducing generalized stimulation/activation of T cells in culture. In some embodiments, the protein complex can increase the T cell proliferation by at least 10%, 20%, 30%, 40%, 50%, 60%, 70%, 80%, 90%, or 100% than control molecules used herein or combinations thereof. In some embodiments, the protein complex described herein can increase cytokine (e.g., IFN-γ and/or IL-2) production by at least 1-fold, 2-fold, 5-fold, 10-fold, 20-fold, 50-fold, 100-fold, 200-fold, 500-fold, 1000-fold, 2000-fold, or 10000-fold than control molecules used herein or combinations thereof.

In some embodiments, the protein complex described herein does not induce cytokine storm in human. In some embodiments, the protein complex described herein is not a superagonist. Details of cytokine storm and superagonist can be found, e.g., in Shimabukuro-Vornhagen, A. et al. “Cytokine release syndrome.” Journal for ImmunoTherapy of Cancer 6.1 (2018): 1-14, which is incorporated herein by reference in its entirety.

In some embodiments, the protein complex described herein can inhibit tumor growth.

### Methods of Making Protein Complexes

Variants of the protein complexes described herein can be prepared by introducing appropriate nucleotide changes into the DNA encoding a polypeptide or a part thereof or by peptide synthesis. Such variants include, for example, deletions, insertions, or substitutions of residues within the amino acids sequences.

Screening can be performed to increase binding affinity of the CD47-binding domains and PD-L1-binding domains. Any combination of deletions, insertions, and/or combinations can be made to arrive at a variant that has increased binding affinity for the target. The amino acid changes introduced into the variant can also alter or introduce new post-translational modifications into the polypeptide, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

The CD47-binding domains and/or PD-L1-binding domains can be derived from any species of animal, including mammals. Non-limiting examples of binding domain variants include sequences derived from humans, primates, e.g., monkeys and apes, cows, pigs, horses, sheep, camelids (e.g., camels and llamas), chicken, goats, and rodents (e.g., rats, mice, hamsters and rabbits).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. Techniques for incorporating DNA into such expression systems are well known to those of ordinary skill in the art. The DNA may also be “naked.” The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be affected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding a polypeptide of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptides can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The protein constructs or polypeptides of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of protein constructs or polypeptides disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the protein constructs or the polypeptides, vector comprising the polynucleotide encoding the protein constructs or the polypeptides, and/or compositions thereof is to be administered, a severity of symptoms and a route of administration, and thus administration can be determined on an individual basis.

An effective amount can be administered in one or more administrations. By way of example, an effective amount of the protein constructs or the polypeptides is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the protein constructs or the polypeptides used.

Effective amounts and schedules for administering the protein constructs or the polypeptides, the polynucleotides encoding the protein constructs or the polypeptides, and/or compositions disclosed herein may be determined empirically, and making such determinations is within the skill in the art. Those skilled in the art will understand that the dosage that must be administered will vary depending on, for example, the mammal that will receive the protein constructs or the polypeptides, the polynucleotides, and/or compositions disclosed herein, the route of administration, the particular type of polynucleotides, and/or compositions disclosed herein used and other drugs being administered to the mammal.

A typical daily dosage of an effective amount of the protein constructs and/or the polypeptides is 0.1 mg/kg to 100 mg/kg (mg per kg of patient weight). In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the dosage is about 1 to 10 mg/kg, about 1 to 5 mg/kg, or about 2 to 5 mg/kg.

In any of the methods described herein, the protein constructs or the polypeptides can be administered to the subject at least once a week (e.g., once a week, twice a week, three times a week, four times a week, once a day, twice a day, or three times a day).

In some embodiments, the one or more additional therapeutic agents can be administered to the subject prior to, or after administering the protein constructs or the polypeptides. In some embodiments, the one or more additional therapeutic agents are administered to the subject such that there is an overlap in the bioactive period of the one or more additional therapeutic agents and the protein constructs or the polypeptides in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-SIRPα antibody, an anti-CD47 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CD20 antibody (e.g., rituximab) or an anti-EGF receptor antibody (e.g., cetuximab).

### Pharmaceutical Compositions and Routes of Administration

Also provided herein are pharmaceutical compositions that contain the protein constructs or the polypeptides described herein. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the agents can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing the protein constructs or the polypeptides described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the agents can be formulated in aqueous solutions, preferably in physiologically-compatible buffers to reduce discomfort at the site of injection. The solution can contain formulatory agents such as suspending, stabilizing and/or dispersing agents. Alternatively the protein constructs or the polypeptides can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the protein constructs or the polypeptides described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents can vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life in vivo.

The pharmaceutical compositions can be included in a container, pack, or dispenser together with instructions for administration. The disclosure also provides methods of manufacturing the protein constructs or the polypeptides for various uses as described herein.

### EXAMPLES

The invention is further described in the following examples, which do not limit the scope of the invention described in the claims.

### Example 1. Design of Fc-Based Designer Biologics (FBDB™) with PD1×SIRPα×TGFβ Trap Formats

Different triple-targeting formats of FBDB™ were designed and developed to (1) direct the therapeutic to PD-L1⁺ tumor cells; (2) combine the power of innate immunity (e.g., targeting the SIRPα/CD47 pathway) and adaptive immunity (e.g., targeting the PD-1/PD-L1 pathway); (3) reverse TGFβ-induced immunosuppression.

Each format contains at least three different types of immune modules that are directly or indirectly connected to the Fc region of an IgG (e.g., human IgG4). The three immune modules are: (1) one or more SIRPα extracellular domains that can stimulate the antigen-presentation by inducing phagocytosis; (2) one or more PD-1 extracellular domains that can block the PD-1/PD-L1 pathway to enhance the T cell function and guide the SIRPα×PD-1×TGFβ-trap molecules to the PD-L1-expressing tumors; and (3) one or more TGFβ-trap molecules (e.g., the extracellular domain of TGF-beta receptor type-2 (TGFBR2)) that can capture the immune-suppressive TGFβ and improve the tumor microenvironment to augment the immune response.

For example, the SIRPα extracellular domains can release the “don't eat me” brake on macrophages by blocking the interaction between CD47 on tumor cells and SIRPα on macrophages; the PD-1 extracellular domains can release the inhibitory brake on effector T cells by blocking the interaction between PD-1 and PD-L1; and the TGFβ-trap molecule can inhibit TGFβ functions in tumor microenvironment (TME).

Four triple-targeting formats of FBDB™ were designed as shown in FIGS. 1A-1D. HCB301-4 (or PST_v2; schematic structure shown in FIG. 1A) includes two identical polypeptide chains, and each polypeptide chain has an amino acid sequence as set forth in SEQ ID NO: 1. Specifically, each polypeptide chain includes, from N-terminus to C-terminus, a PD-1 extracellular domain (SEQ ID NO: 5; with sequence identical to amino acids 26-170 of human PD-1 protein (NCBI Accession No.: NP_005009.2; SEQ ID NO: 11)), a SIRPα extracellular domain (SEQ ID NO: 6; with sequence identical to amino acids 31-148 of human SIRPα protein (NCBI Accession No.: AAH26692.1; SEQ ID NO: 12)), a human IgG4 hinge region (with S228P mutation according to EU numbering), a human IgG4 Fc, and a TGFBR2 extracellular domain (SEQ ID NO: 7; with sequence identical to amino acids 24-159 of NCBI Accession No.: NP_003233.4; SEQ ID NO: 13)). The PD-1 extracellular domain and the SIRPα extracellular domain are connected via a (GSG) 6 linker peptide (SEQ ID NO: 8). The TGFBR2 extracellular domain is connected to the C-terminus of the human IgG4 Fc via a (G4S) 4G linker peptide (SEQ ID NO: 9).

HCB301-3 (or SPT_v2; schematic structure shown in FIG. 1B) includes two identical polypeptide chains, and each polypeptide chain has an amino acid sequence as set forth in SEQ ID NO: 2. Specifically, each polypeptide chain includes, from N-terminus to C-terminus, a SIRPα extracellular domain (SEQ ID NO: 6), a PD-1 extracellular domain (SEQ ID NO: 5, a human IgG4 hinge region (with S228P mutation according to EU numbering), a human IgG4 Fc, and a TGFBR2 extracellular domain (SEQ ID NO: 7). The SIRPα extracellular domain and the PD-1 extracellular domain are connected via a (GSG) 6 linker peptide (SEQ ID NO: 8). The TGFBR2 extracellular domain is connected to the C-terminus of the human IgG4 Fc via a (G4S) 4G linker peptide (SEQ ID NO: 9).

HCB301-2 (or P2ST_v2; schematic structure shown in FIG. 1C) includes two identical polypeptide chains, and each polypeptide chain has an amino acid sequence as set forth in SEQ ID NO: 3. Specifically, each polypeptide chain includes, from N-terminus to C-terminus, two identical PD-1 extracellular domains (SEQ ID NO: 5) that are linked via a (G4S) 3 linker (SEQ ID NO: 10), a SIRPα extracellular domain (SEQ ID NO: 6), a human IgG4 hinge region (with S228P mutation according to EU numbering), a human IgG4 Fc, and a TGFBR2 extracellular domain (SEQ ID NO: 7). The two PD-1 extracellular domains are connected to the N-terminus of the SIRPα extracellular domain via a (GSG) 6 linker peptide (SEQ ID NO: 8). The TGFBR2 extracellular domain is connected to the C-terminus of the human IgG4 Fc via a (G4S) 4G linker peptide (SEQ ID NO: 9).

HCB301-1 (or SP2T_v2; schematic structure shown in FIG. 1D) includes two identical polypeptide chains, and each polypeptide chain has an amino acid sequence as set forth in SEQ ID NO: 4. Specifically, each polypeptide chain includes, from N-terminus to C-terminus, a SIRPα extracellular domain (SEQ ID NO: 6), two identical PD-1 extracellular domains (SEQ ID NO: 5) that are linked via a (G4S) 3 linker (SEQ ID NO: 10), a human IgG4 hinge region (with S228P mutation according to EU numbering), a human IgG4 Fc, and a TGFBR2 extracellular domain (SEQ ID NO: 7). The SIRPα extracellular domain is connected to one of the two PD-1 extracellular domains via a (GSG) 6 linker peptide (SEQ ID NO: 8). The TGFBR2 extracellular domain is connected to the C-terminus of the human IgG4 Fc via a (G4S) 4G linker peptide (SEQ ID NO: 9).

The expressed proteins were purified by a protein A column, followed by HPLC-SEC (high-performance liquid chromatography coupled with size-exclusion chromatography; Agilent), and the percentage of high molecular weight peaks (HMW %), the percentage of the main peak (Main %), and the percentage of low molecular weight peaks (LMW %) were measured.

Specifically, the HCB301 proteins were expressed in CHO—S cells. The culture supernatant was collected and subject to protein A purification. The culture supernatant pH was adjusted to 8.0 by adding a concentrated equilibration buffer (250 mM Tris, 1500 mM NaCl, pH 8.0). Next, the protein A column was equilibrated with 10× column volume of an equilibration buffer (25 mM Tris, 150 mM NaCl, pH 8.0), and the culture supernatant was then loaded to the equilibrated protein A column. The column was then washed with 6× column volume of a wash buffer (100 mM citric acid, 500 mM NaCl). The protein sample was eluted by 6× column volume of an elution buffer (100 mM acetate, 200 mM NaCl, pH 3.0), and pH was adjusted to 6.5-7 by a buffer containing 1 M Hepes, pH 8.0. As shown in the table below, the expression level (titer) of HCB301-3 and HCB301-4 were comparable and higher than that HCB301-1 or HCB302-2. After protein A purification, the percentage of the main peak (Main %) of the HCB301 proteins ranged from 87.88% to 94.38%; the percentage of high molecular weight peaks (HMW %) of the HCB301 proteins ranged from 1.35% to 5.43; and the percentage of low molecular weight peaks (LMW %) of the HCB301 proteins ranged from 1.27% to 10.78%. The results indicate that all four HCB301 proteins (HCB301-1, HCB301-2, HCB301-3, and HCB301-4) can be expressed and harvested with a high purity.

TABLE 1

| | | | | HPLC-SEC (Agilent) profiles | | |
|---|---|---|---|---|---|---|
| | | | | of HCB301 (CHO-S) after | | |
| | | | | Protein A purification | | |
| | MW | | Titer | HMW | Major | LMW |
| Format | (kDa) | pI | (mg/mL) | % | % | % |
| HCB301-4 | 145.11 | 5.77 | 177.15 | 4.35 | 94.38 | 1.27 |
| HCB301-3 | 145.11 | 5.77 | 179.87 | 5.43 | 92.35 | 2.22 |
| HCB301-2 | 179.27 | 6.05 | 62.07 | 1.35 | 87.88 | 10.78 |
| HCB301-1 | 179.27 | 6.05 | 109.21 | 2.57 | 91.22 | 6.22 |

In addition, the amino acid sequences of HCB301-1, HCB301-2, HCB301-3, and HCB301-4 were analyzed using the deimmunization tool (Immune Epitope Database And Analysis Resource; Dhanda et al. “Development of a strategy and computational application to select candidate protein analogues with reduced HLA binding and immunogenicity.” Immunology 153.1 (2018): 118-132) to identify immunogenic regions. No immunogenicity was identified.

### Example 2. Determination of the Whole Cell Binding Ability to CD47 tf CHO—S Cells

To determine the whole cell binding ability of the HCB301 proteins to CD47 expressed on cell surface, transfected CHO—S cells expressing human CD47 (CD47 tf CHO—S) were used as target cells. 5×10⁴ cells were incubated with serially diluted HCB301 proteins at indicated concentrations (6.4 pM, 32 pM, 160 pM, 0.8 nM, 4 nM, 20 nM, 100 nM, and 500 nM) in FACS buffer (phosphate-buffered saline (PBS) supplemented with 4% fetal bovine serum (FBS)) at 4° C. for 30 minutes. After the incubation, the cells were washed twice with FACS buffer, and then incubated with R-Phycoerythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA). SIRPα_G4 was used as a positive control. SIRPα_G4 includes two identical polypeptide chains, and each polypeptide chain has an amino acid sequence as set forth in SEQ ID NO: 14. PD1_G4 was used as a negative control. PD1_G4 includes two identical polypeptide chains, and each polypeptide chain has an amino acid sequence as set forth in SEQ ID NO: 15.

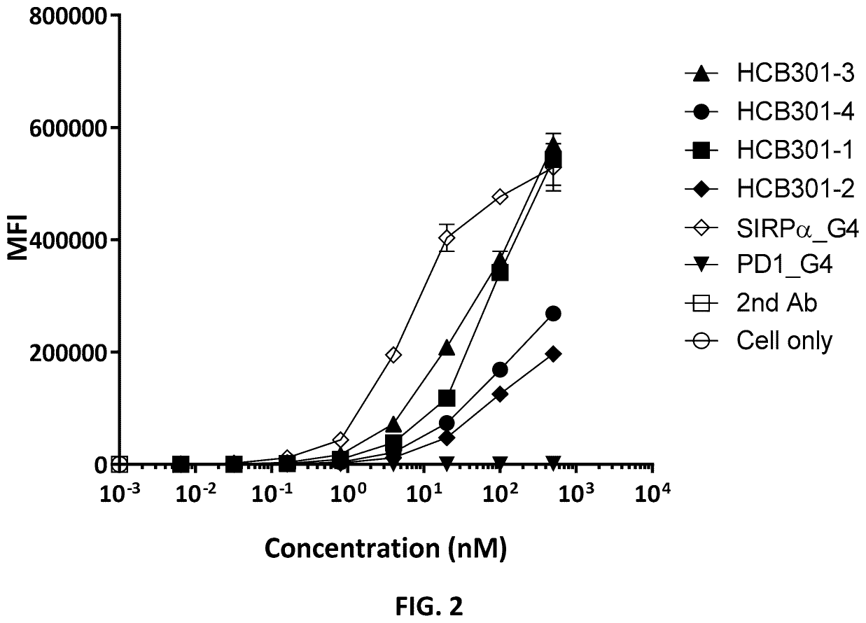

As shown in FIG. 2, all four HCB301 proteins can bind to CD47 tf CHO—S cells. Specifically, the positive control SIRPα_G4 exhibited the highest CD47 binding ability, followed by HCB301-3 and HCB301-1. HCB301-4 and HCB301-2 can also bind to CD47-expressing cells. By contrast, no binding signals were detected for the negative control PD1_G4.

### Example 3. Determination of the Whole Cell Binding Ability to PDL1 tf CHO—S Cells

To determine the binding ability of the HCB301 proteins to PD-L1, transfected CHO—S cells expressing human PD-L1 (PDL1 tf CHO—S) were used as target cells. 3×10⁴ cells were incubated with serially diluted HCB301 proteins at indicated concentrations (6.4 pM, 32 pM, 160 pM, 0.8 nM, 4 nM, 20 nM, and 100 nM) in FACS buffer (PBS supplemented with 4% FBS) at 4° C. for 30 minutes. After the incubation, the cells were washed twice with FACS buffer, and then incubated with R-Phycoerythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA). PD1_G4 was used as a positive control. Hu5F9-G4 (an anti-CD47 reference antibody) and SIRPα_G4 were used as negative controls.

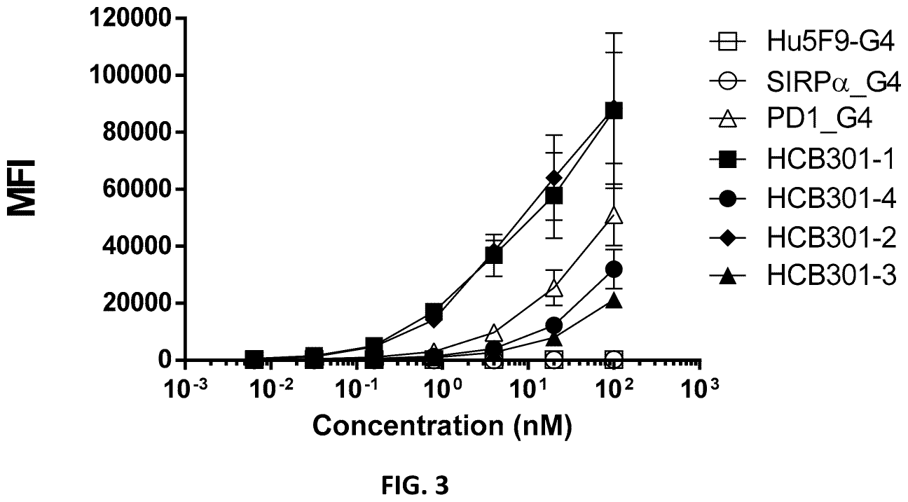

As shown in FIG. 3, all four HCB301 proteins can bind to PDL1 tf CHO—S cells. Specifically, HCB301-1 and HCB301-2 exhibited a higher binding ability than the positive control PD1_G4, and PD1_G4 exhibited a higher binding ability than HCB301-4 and HCB301-3. By contrast, no binding signals were detected for the negative controls Hu5F9-G4 and SIRPα G4.

### Example 4. Determination of the Whole Cell Binding Ability to CD47-Expressing Tumor Cells

To determine the whole cell binding ability of HCB301 proteins to CD47 expressed on tumor cell surface, hypopharyngeal carcinoma FaDu cells expressing endogenous CD47 were used as target cells. 3×10⁴ FaDu cells were incubated with serially diluted HCB301 proteins at indicated concentrations (6.4 pM, 32 pM, 160 pM, 0.8 nM, 4 nM, 20 nM, and 100 nM) in FACS buffer (PBS supplemented with 4% FBS) at 4° C. for 30 minutes. After the incubation, the cells were washed twice with FACS buffer, and then incubated with R-Phycocrythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA). Hu5F9-G4 and SIRPα_G4 were used as positive controls. PD1_G4 was used as a negative control.

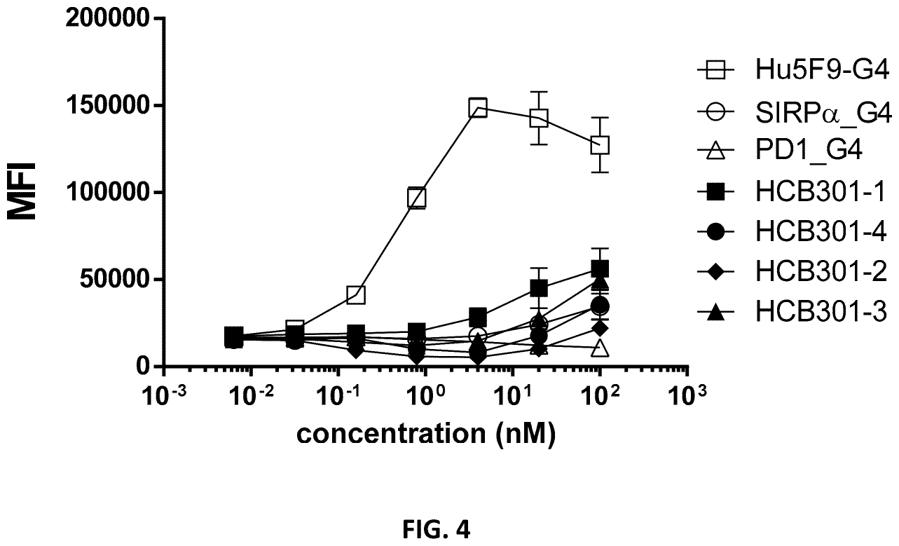

As shown in FIG. 4, all four HCB301 proteins can bind to FaDu cells. Specifically, HCB301-1 showed the highest binding ability, followed by HCB301-3, HCB301-4, and HCB301-2. By contrast, no binding signal was detected for the negative control PD1_G4.

### Example 5. Determination of the Whole Cell Binding Ability to RBCs

To determine the binding ability of HCB301 proteins to red blood cells (RBCs), 1×10⁵ human RBCs were incubated with serially diluted HCB301 proteins at indicated concentrations (6.4 pM, 32 pM, 160 pM, 0.8 nM, 4 nM, 20 nM, 100 nM, and 500 nM) in FACS buffer (PBS supplemented with 4% FBS) at 4° C. for 30 minutes. After the incubation, the RBCs were washed twice with FACS buffer, and then incubated with R-Phycoerythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA). Hu5F9-G4 was used as a positive control. PD1_G4 was used as negative controls.

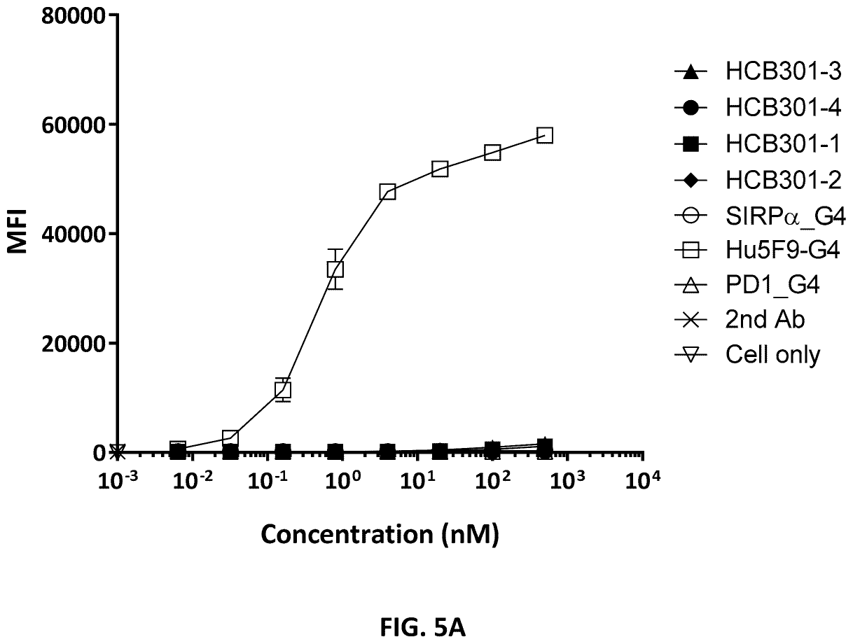

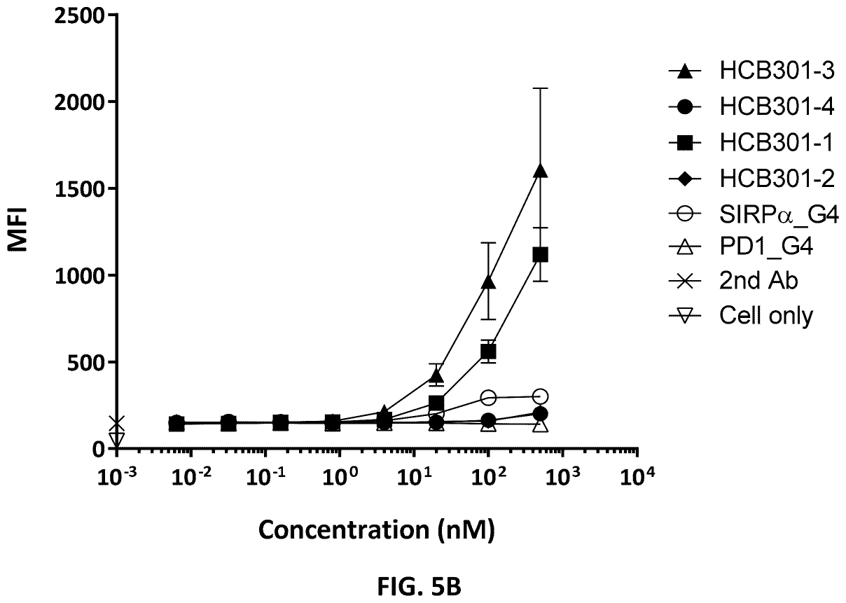

As shown in FIGS. 5A-5B, the positive control Hu5F9-G4 exhibited a strong binding ability to RBCs. By contrast, all four HCB301 proteins showed weak binding to RBCs. Specifically, HCB301-3 showed a higher RBC binding ability than HCB301-1, and the binding ability of both HCB301-3 and HCB301-1 was higher than that of SIRPα_G4. HCB301-4 and HCB301-2 showed a lower RBC binding ability, which was comparable to that of PD1_G4.

### Example 6. Determination of the Whole Cell Binding Ability to Platelets

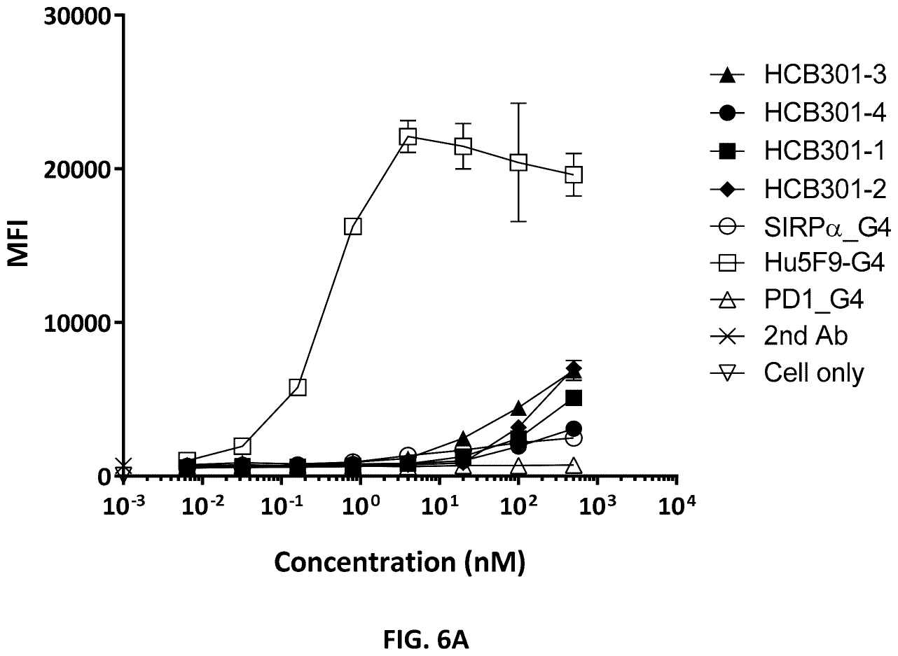

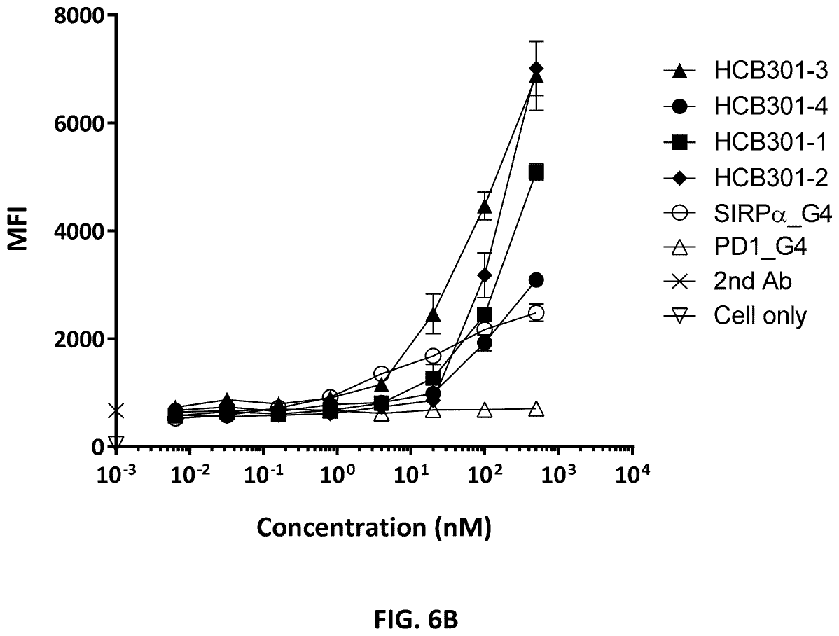

To determine the binding ability of HCB301 proteins to platelets, 5×10⁵ human platelets were incubated with serially diluted HCB301 proteins at indicated concentrations (6.4 pM, 32 pM, 160 pM, 0.8 nM, 4 nM, 20 nM, 100 nM, and 500 nM) in FACS buffer (PBS supplemented with 4% FBS) at 4° C. for 30 minutes. After the incubation, the platelets were washed twice with FACS buffer, and then incubated with R-Phycoerythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA). Hu5F9-G4 was used as a positive control. PD1_G4 and SIRPα_G4 were used as negative controls. As shown in FIGS. 6A-6B, the positive control Hu5F9-G4 exhibited a strong binding ability to platelets. By contrast, all HCB301 proteins showed weak binding to platelets. Specifically, HCB301-3 showed a higher platelet binding ability than HCB301-2 and HCB301-1. HCB301-4 showed a lower RBC binding ability, which was comparable to that of SIRPα_G4.

### Example 7. Determination of the Binding Ability to hTGFβ1, hTGFβ2, and hTGFβ3

To determine the binding ability of HCB301 proteins to human TGFβ, a binding titration ELISA assay was performed using of the HCB301 proteins at indicated concentrations (25.4 pM, 76.2 pM, 228.6 pM, 685.8 pM, 2.1 nM, 6.2 nM, 18.5 nM, 55.6 nM, 166.7 nM, or 500 nM). IgG4-TGFβ trap was used as a positive control. SIRPα_G4 was used as a negative control. A 96-well EIA microplate was coated with 1 μg/ml human TGFβ (TGFβ1, TGFβ2, or TGFβ3) overnight at 4° C. After blocking with 1×PBS containing 5% skim milk, diluted HCB301 proteins were added and incubated at room temperature (RT) for 1 hour. The unbound proteins were removed by washing the wells with 1×PBST (1×PBS containing 0.1% Tween 20) three times. A HRP-conjugated secondary antibody (1:5000) was added to the wells at RT for 1 hour. After the incubation, excess secondary antibodies were removed by washing the wells with 1× PBST three times. Finally, 3,3′,5,5′-Tetramethylbenzidine (TMB) was added to the color development. The reaction was stopped and HRP activity was measured using a spectrophotometer at 450 nm.

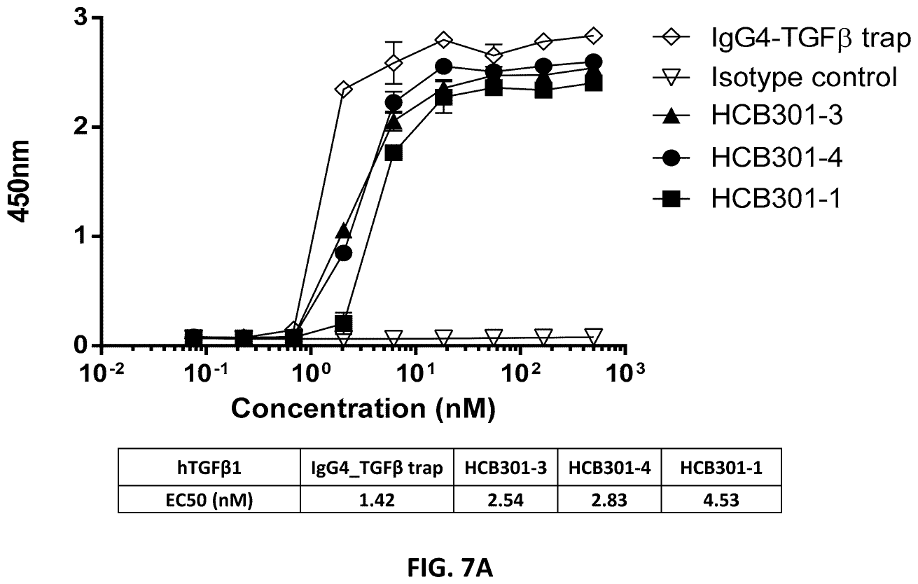

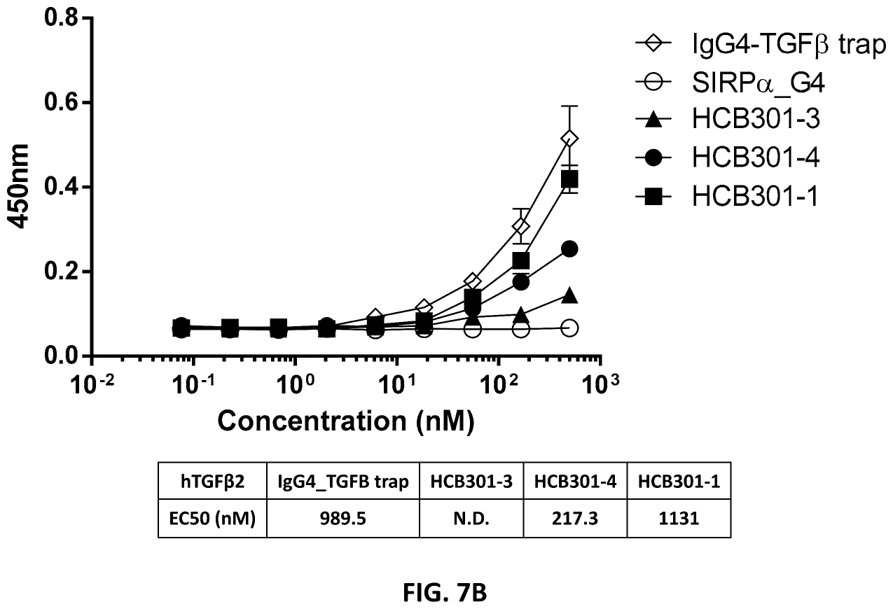

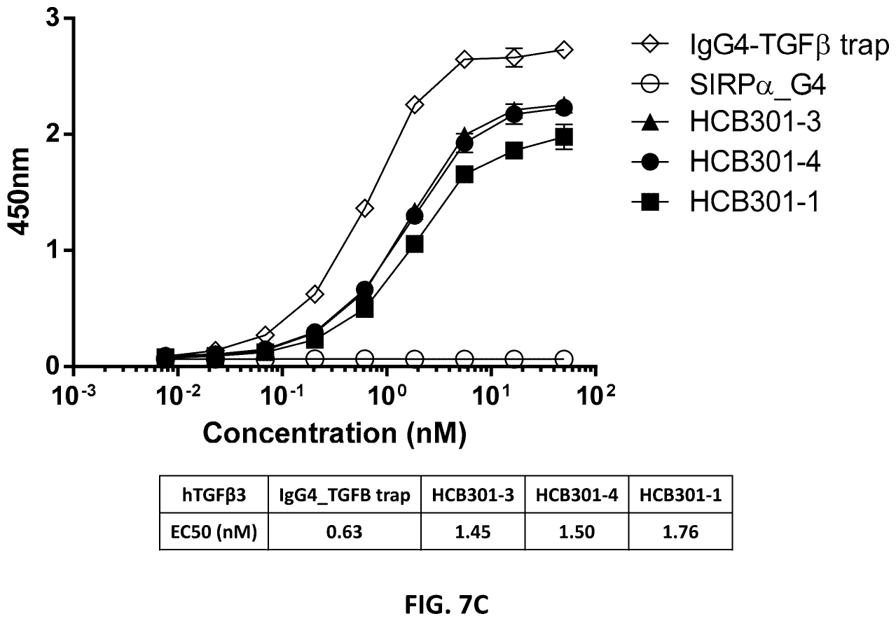

As shown in FIGS. 7A-7C, the EC50 values were also determined according to the binding curves. The results indicate that HCB301 proteins can bind to hTGFβ1 and hTGFβ3 but showed weak binding ability to hTGFβ2. Specifically, the EC50 values of HCB301-1, HCB301-3, and HCB301-4 binding to hTGFβ3 was lower than their respective EC50 values binding to hTGFβ1.

### Example 8. Selective Binding to PD-L1-Expressing Cells

The selective binding ability of HCB301 proteins to PD-L1-expressing cells was determined as follows. OE19 cells were labeled with CellTrace™ CFSE (Thermo, Cat #: C34554) and transfected OE19 cells expressing PD-L1 (PD-L1 tf OE19) were labeled with Celltrace™ violet (Thermo, Cat #: C34557) according to the instructions provided by manufacturer. 2×10⁴ cells/well of CellTrace™ CFSE-labeled OE19 cells (OE19-CFSE) and 2×10⁴ cells/well of CellTrace™ violet-labeled transfected OE19 cells expressing PD-L1 tf OE19 cells (OE19-violet) were incubated with serially diluted HCB301 proteins at indicated concentrations (6.4 pM, 32 pM, 160 pM, 0.8 nM, 4 nM, 20 nM, 100 nM, and 500 nM) in FACS buffer (PBS supplemented with 4% FBS) at 4° C. for 30 minutes. After the incubation, the cells were washed twice with FACS buffer, and then incubated with R-Phycocrythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA). Afterwards, the cells were gated according to PE signals to calculate the percentages of different cell types by Kaluza analysis software (Beckman Coulter Inc.). PD1_G4 was used as a positive control and SIRPα_G4 was used as a negative control.

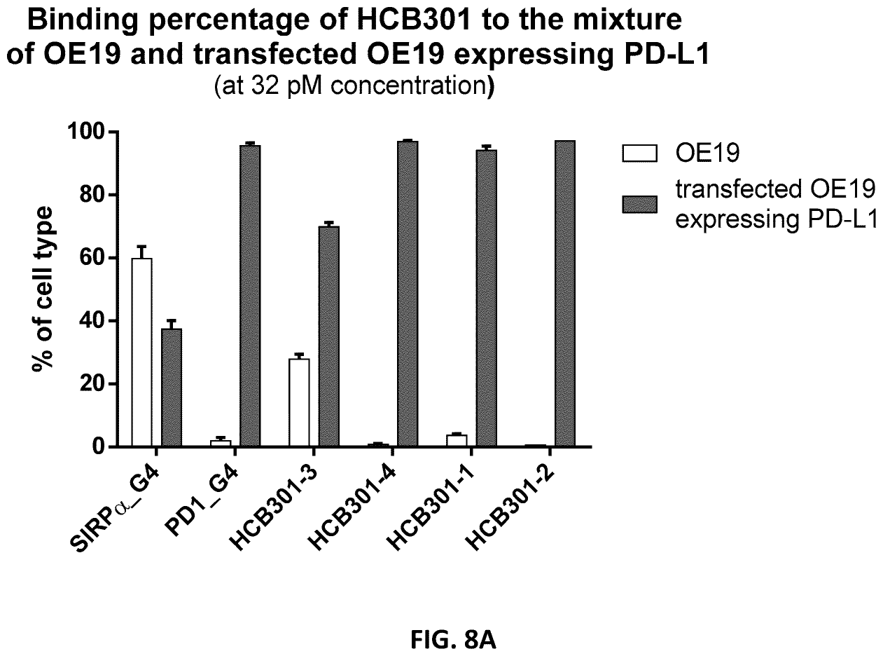

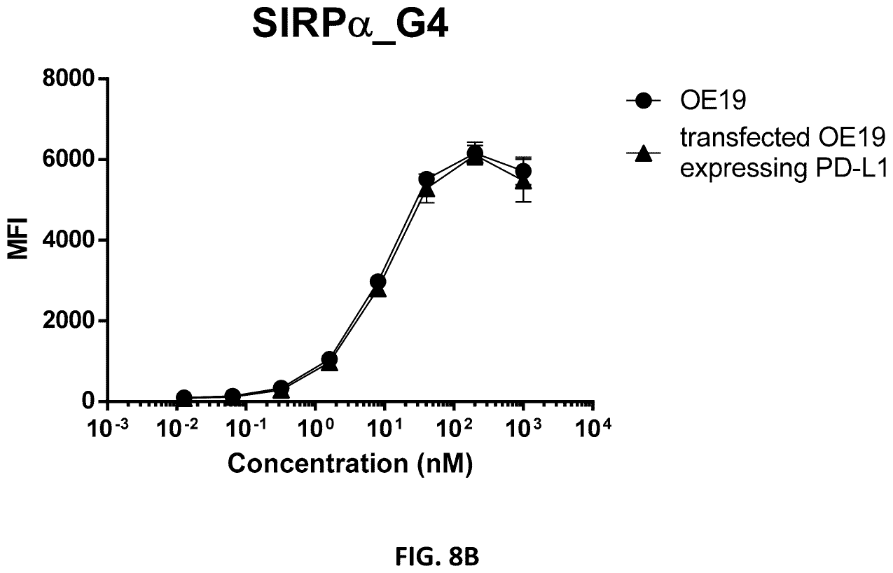

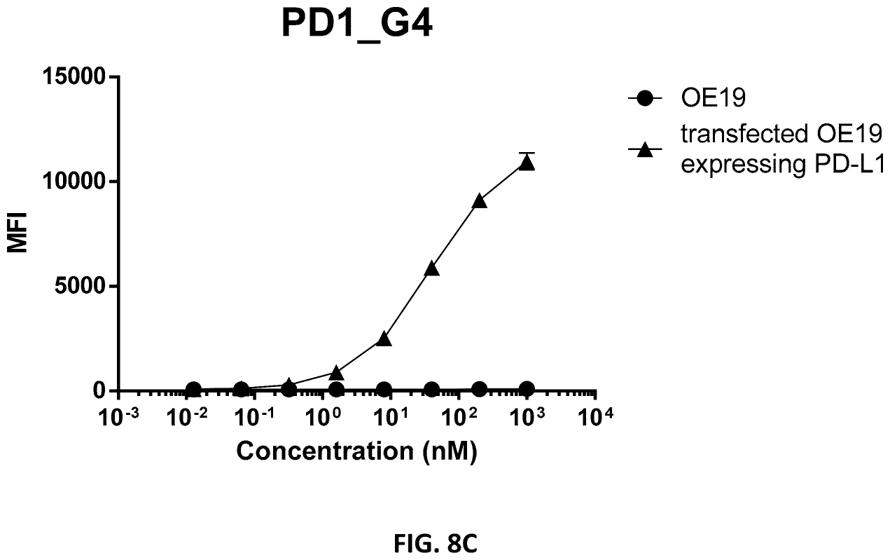

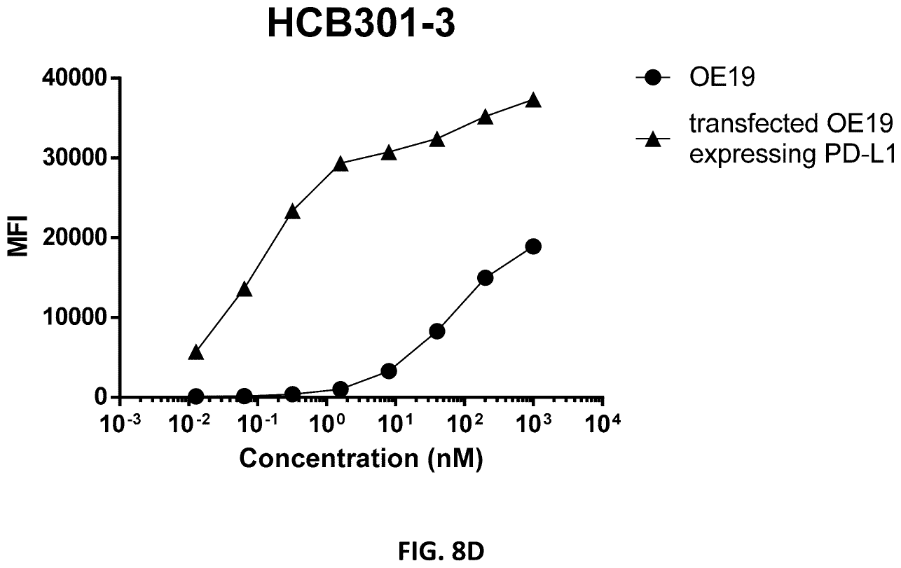

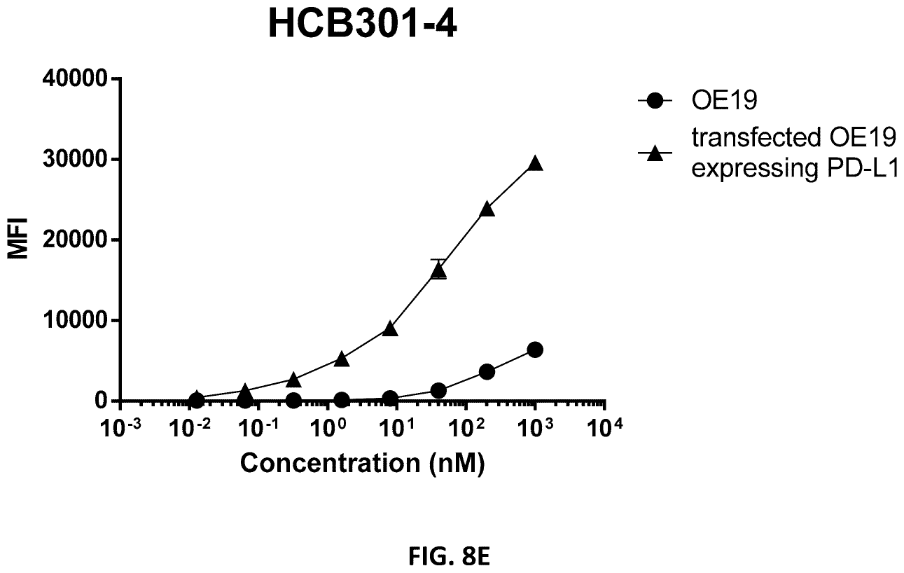

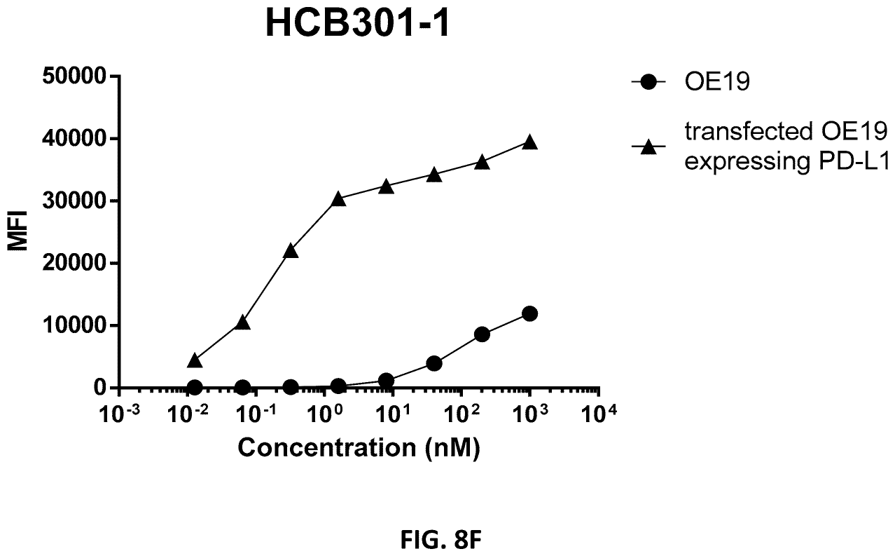

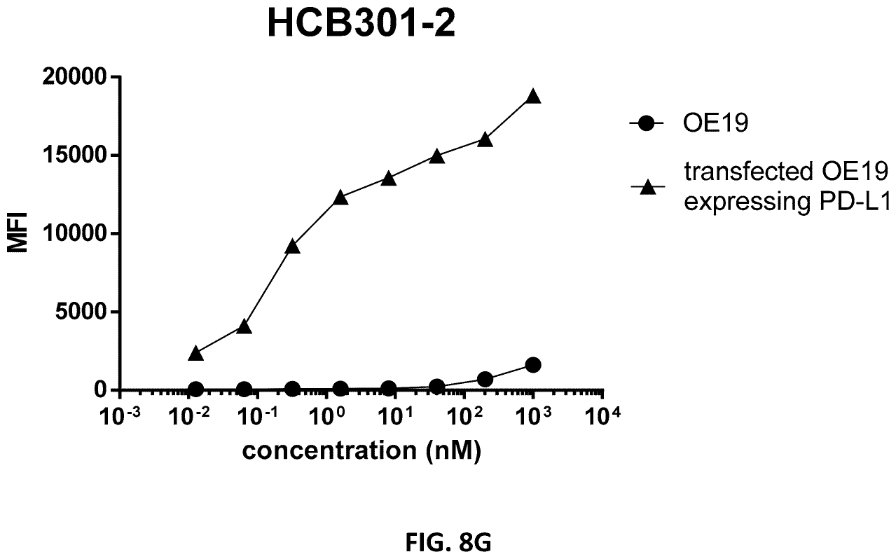

As shown in FIG. 8A, at 32 pM, all HCB301 proteins showed selective binding to OE19-violet cells expressing PD-L1 over the OE19-CFSE cells. FIGS. 8B-8G show the binding curves of SIRPα_G4, PD1_G4, and the four HCB301 proteins (HCB301-1, HCB301-2, HCB301-3 and HCB301-4) to the two cell types, respectively.

### Example 9. Hemagglutination (HA) Activity

To determine the HA activity induced by HCB301 proteins, a 10% RBC solution was prepared from whole blood of a healthy donor. The RBCs were washed with a 0.9% NaCl buffer twice, and then diluted to 10% by volume in a 0.9% NaCl buffer. The HCB301 proteins were serially diluted (3-fold) to a final concentration of 8.4 pM, 25.4 pM, 76.2 pM, 228.6 pM, 685.8 pM, 2.1 nM, 6.2 nM, 18.5 nM, 55.6 nM, 166.7 nM, or 500 nM. The diluted proteins were incubated in a round-bottom 96-well plate with the 12 μl of the 10% RBC solution at RT overnight. Agglutinated RBCs coated the wells evenly, whereas non-agglutinated cells formed a distinct red dot at the bottom of the wells. Hu5F9-G4 was used a positive control PC. SIRPα_G4, PD1_G4, and IgG4-TGFβ trap were used as negative controls.

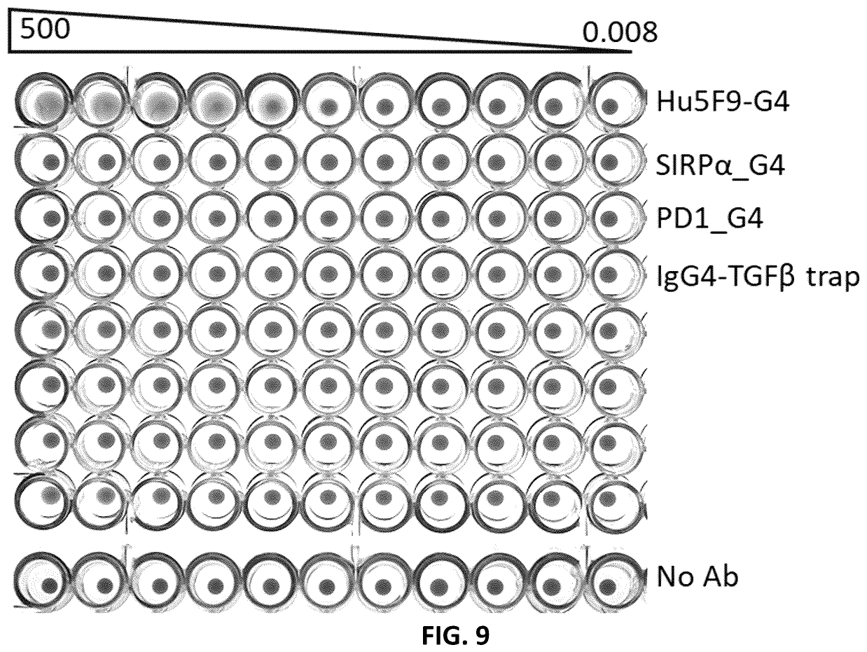

An image of the plate was captured on the next day, as shown in FIG. 9. The image indicates that only Hu5F9-G4 induced HA activity at high concentrations, whereas none of the other tested molecules, including the four HCB301 proteins, induced HA activity within the above indicated concentration range.

### Example 10. Determination of the Blocking Effect to SIRPα/CD47 Interaction

To determine the SIRPα ligand blocking ability of HCB301 proteins, flow cytometry-based assays were performed using CD47 tf CHO—S cells as the target cells. Specifically, 3×10⁴ cells/well of CD47 tf CHO—S cells were incubated with the HCB301 proteins were serially diluted at indicated concentrations (0.2 pM, 1.9 pM, 15.2 pM, 122.1 pM, 876.6 pM, 7.8 nM, 62.5 nM, and 500 nM) in FACS buffer (PBS supplemented with 4% FBS), together with a fixed concentration of biotinylated SIRPα_G4 at 4° C. for 30 minutes. After washing, streptavidin-PE (eBioscience, Cat #: EBS12-4317-87) was added at 0.3 μg per well, and PE signals from the cells were analyzed using a CytoFLEX flow cytometer (Beckman Coulter Inc.). Hu5F9 and SIRPα_G4 were used as positive controls. An anti-PD-1 antibody (with human IgG4 Fc) was used as an isotype control.

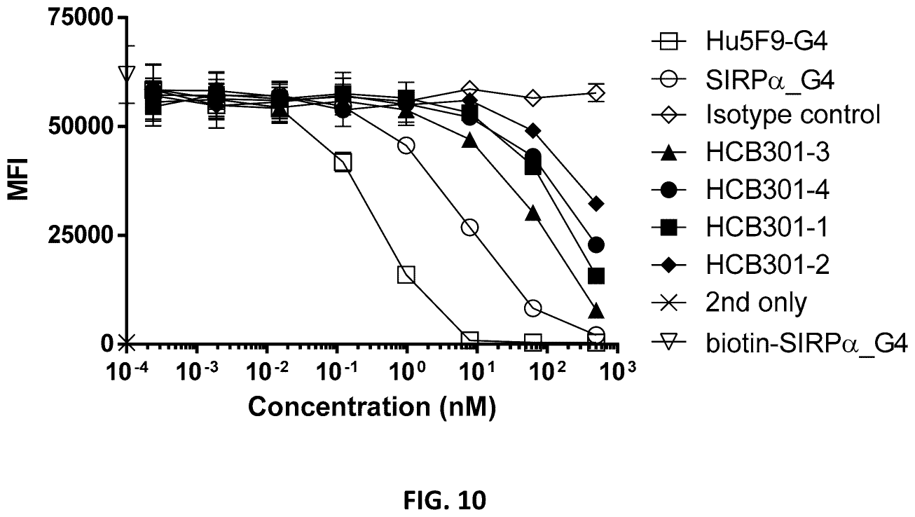

As shown in FIG. 10, Hu5F9-G4 exhibited the strongest blocking effect, followed by SIRPα_G4, and the four HCB301 proteins. More specifically, the blocking effect of the four HCB301 proteins can be ranked from strong to weak as: HCB301-3, HCB301-1, HCB301-4, and HCB301-2.

### Example 11. Determination of the Blocking Effect to PD-1/PD-L1 Interaction

The blocking effect of HCB301 proteins to the interaction between PD-1 and PD-L1 tf CHO—S cells was determined as follows. 3×10⁴ cells/well of PD-L1 tf CHO—S cells were incubated with the HCB301 proteins that were serially diluted at indicated concentrations (0.2 pM, 1.9 pM, 15.2 pM, 122.1 pM, 876.6 pM, 7.8 nM, 62.5 nM, and 500 nM) in FACS buffer (PBS supplemented with 4% FBS), together with a fixed concentration of biotinylated PD1_G4 at 4° C. for 30 minutes. After washing, streptavidin-PE (eBioscience, Cat #: EBS12-4317-87) was added at 0.3 μg per well, and PE signals from the cells were analyzed using a CytoFLEX flow cytometer (Beckman Coulter Inc.). MPDL3280A (an anti-PD-L1 antibody) and PD1_G4 were used as positive controls.

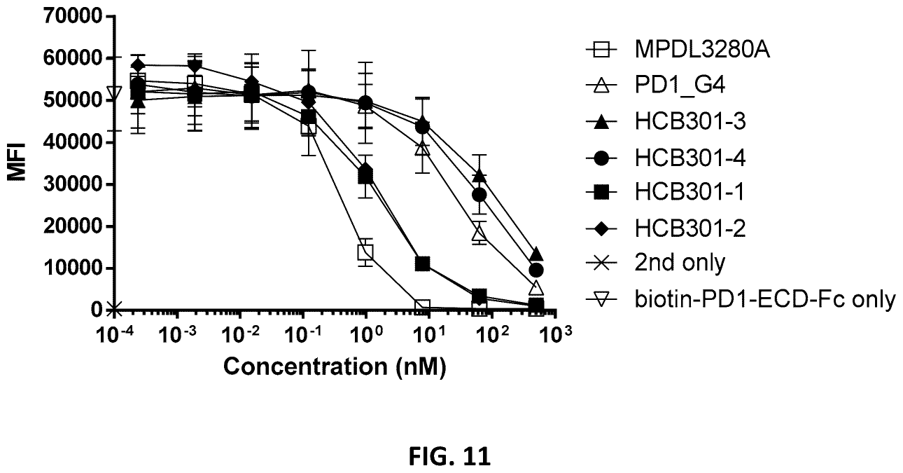

As shown in FIG. 11, MPDL3280A exhibited the strongest blocking effect. HCB301-1 and HCB301-2 showed a stronger blocking effect than that of PD1_G4. HCB301-4 showed a weaker blocking effect than that of PD1_G4, but a stronger blocking effect than HCB301-3. The results indicate that HCB301 proteins having two PD-1 extracellular domains in each polypeptide chain can block the PD-1/PD-L1 interaction more efficiently than those with one PD-1 extracellular domain in each polypeptide chain.

### Example 12. Inhibition of TGFβ-Induced smad2 Reporter Activity

The ability of HCB301 proteins to inhibit TGFβ1-induced smad2 reporter activity was determined as follows. The HCB301 proteins were serially diluted (5-fold) to final concentrations of 6.4 pM, 32 pM, 160 pM, 0.8 nM, 4 nM, 20 nM, 100 nM, and 500 nM. M7824 (an anti-PD-L1×TGFβ trap), anti-TGFβ (an anti-TGFβ antibody, BioXcell, Cat #: BE0057), and IgG4-TGFβ trap were used as positive controls. SIRPα_G4 was used as a negative control. 4×10³ transfected HEK293T cells expressing a smad2 reporter was incubated with the diluted HCB301 proteins, together with 20 ng/ml TGFβ1, TGFβ2, and TGFβ3, respectively. After an 24-hour incubation at 37° C. in a 5% CO₂ incubator, luminescence signals were detected by Varioskan™ LUX multimode microplate reader (Thermo).

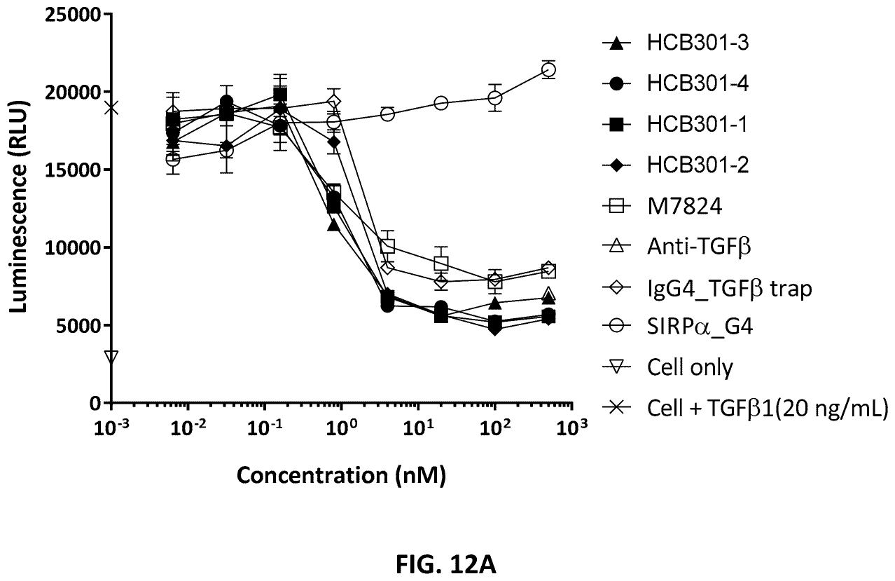

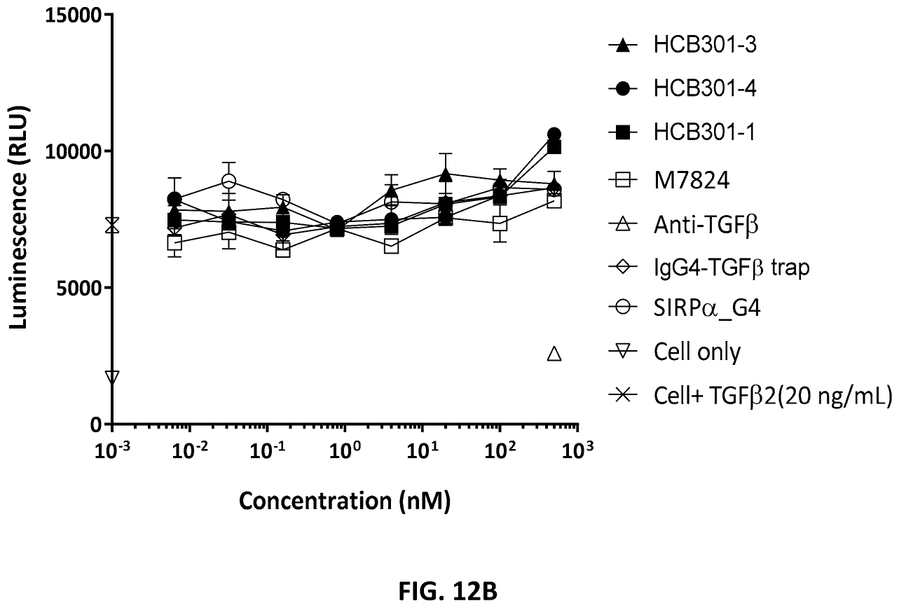

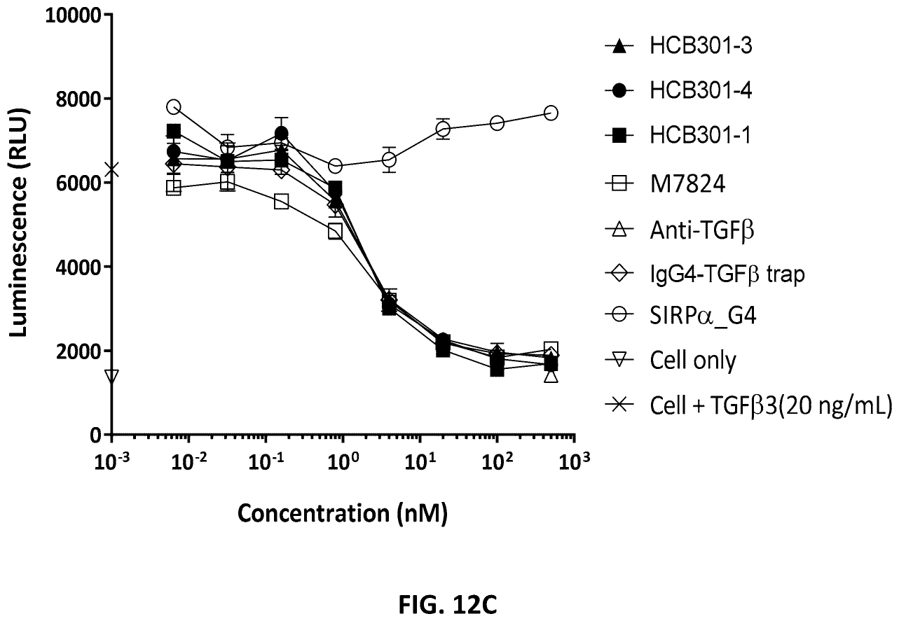

As shown in FIG. 12A, all four HCB301 proteins can inhibit TGFβ1-mediated smad2 reporter activity. Each HCB301 protein showed a stronger inhibition ability than M7824 and IgG4-TGFβ trap. Specifically, HCB301-3, HCB301-4, and HCB301-1 showed a stronger inhibition ability than that of HCB301-2. As shown in FIGS. 12B-12C, HCB301-3, HCB301-4, and HCB301-1 can inhibit TGFβ3-mediated, but not TGFβ2-mediated smad2 reporter activity. In particular, HCB301-3, HCB301-4, and HCB301-1 exhibited a comparable TGFβ3 inhibitory ability as compared to that of IgG4-TGFβ trap or M7824.

### Example 13. Induction of Macrophages to Phagocytose CD47-Expressing Tumor Cells

To determine the macrophage-mediated phagocytosis induced by HCB301 proteins on cancer cells, phagocytosis assay were performed as follows. CD47-expressing Jurkat cells were labeled with 5 nM CellTrace™ CFSE (Thermo, Cat #: C34554) at 37° C. for 10 minutes, and them washed by complete RPMI-1640 medium. 1×10⁵ cells/well CFSE-labeled Jurkat cells (target cells) were incubated with the HCB301 proteins that were serially diluted at indicated concentrations (10 pM, 100 pM, 1 nM, 10 nM, 100 nM, and 1 μM) in a low binding 96-well U-shaped bottom plate at 37° C. for 30 minutes. Afterwards, 5×10⁴ Raw264.7 mouse microphages were added to each well and the plate was incubated at 37° C. for 2 hours. The Raw264.7 cells were stained with PE-Cyanine 7 conjugated F4/80 antibody (eBioscience, Cat #: 25-4801-82). The phagocytosis ability of the HCB301 proteins was evaluated by calculating the percentage of CFSE+F4/80+ from macrophages (indicating macrophages phagocytosed CFSE-labeled Jurkat cells) over the total F4/80 signals from macrophages by a CytoFlex flow cytometer (Beckman Coulter Inc.). Hu5F9-G4 and SIRPα_G4 were used as positive controls. PD1_G4 was used as a negative control. An anti-PD-1 antibody (with human IgG4 Fc) was used as an isotype control.

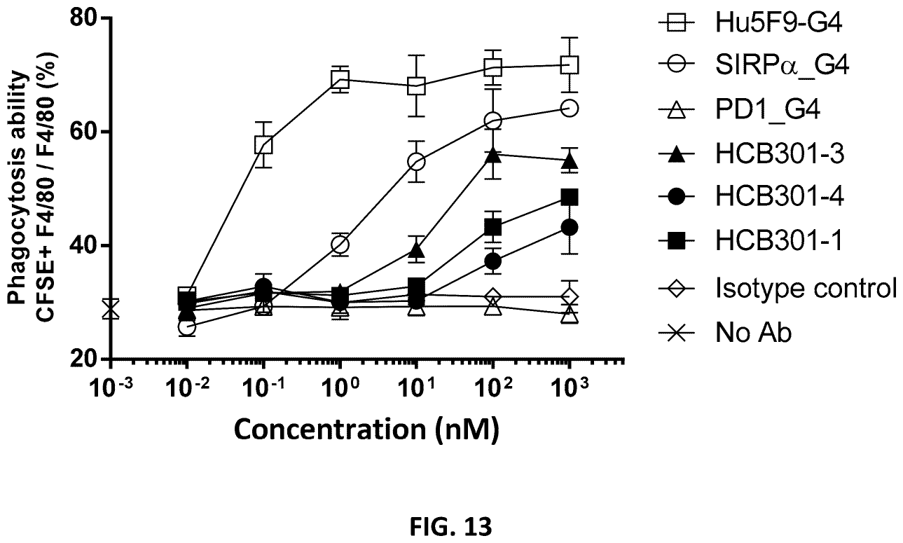

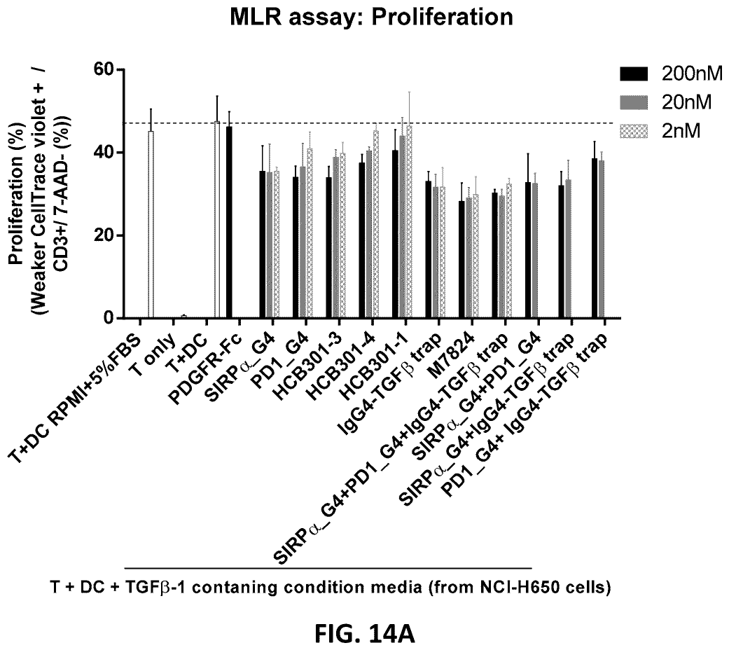

As shown in FIG. 13, Hu5F9-G4 exhibited the strongest ability to induce Raw264.7-mediated phagocytosis on CD47-expressing Jurkat cells, followed by SIRPα_G4. HCB301-3, HCB301-1, and HCB301-4 also induced microphages to phagocytose the Jurkat cells. By contrast, PD1_G4 and the isotype control did not induce phagocytosis at the tested concentrations.

### Example 14. Enhancement of T Cell Response in the MLR Assay in the Presence of TGFβ1

Mixed lymphocyte reaction (MLR) assays in the presence of TGFβ1 were performed to determine the enhancement of T cell response by HCB301 proteins. T cells were labeled with 5 nM CellTrace™ violet (Thermo, Cat #: C34557) at 37° C. for 10 minutes, and then washed with complete RPMI-1640 medium twice. 1×10⁵ CellTrace™ violet-labeled CD4+ T cells and 1×10⁴ dendritic cells (DCs) were incubated with the HCB301 proteins at 2 nM, 20 nM, or 200 nM. Control molecules or combinations thereof were also used for incubation, e.g., PDGFR-Fc, SIRPα G4, PD1_G4, IgG4-TGFβ trap, M7824, SIRPα_G4+PD1_G4+IgG4-TGFβ trap, SIRPα_G4+PD1_G4, SIRPα_G4+IgG4-TGFβ trap, and PD1_G4+IgG4-TGFβ trap. PDGFR-Fc served as a negative control. PDGFR-Fc includes two identical polypeptide chains, and each polypeptide chain has a PDGFR extracellular domain fused to an Fc. TGFβ1-containing condition media from NCl—H650 cells was then added, and co-incubated for 5 days. After the co-incubation, cells were harvested and cell proliferation was analyzed by a CytoFLEX-S flow cytometer (Beckman Coulter Inc.). Culture supernatant were also collected, and IL-2 secretion and IFN-γ secretion were determined using Human IL-2 ELISA MAX Deluxe kit (BioLegend, Cat #: 431805) and Human IFNγ ELISA MAX Deluxe kit (BioLegend, Cat #: 430105), respectively.

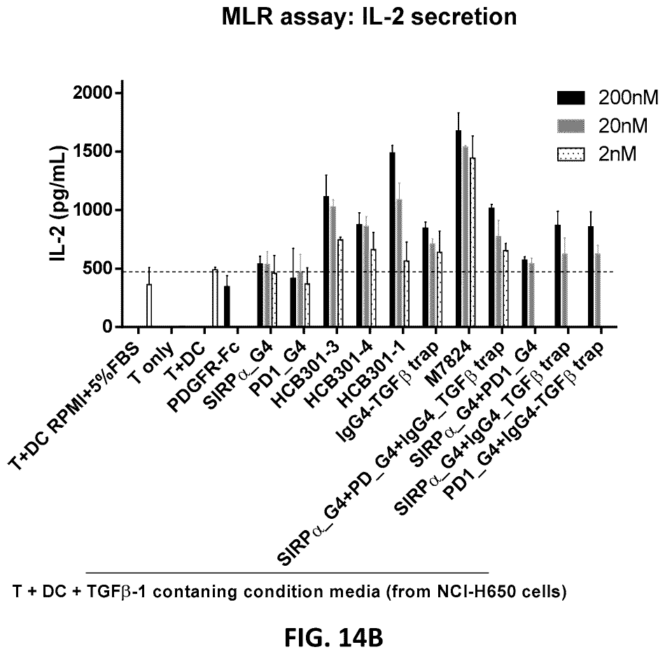

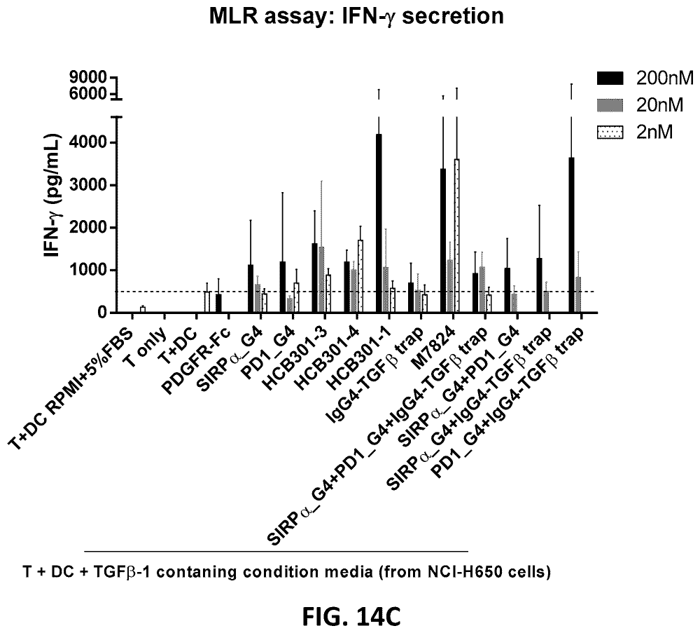

As shown in FIG. 14A, cell proliferation in the MLR assays was determined using the CellTrace™ violet cell proliferation kit (Thermo, Cat #: C34557). Specifically, the percentage of weaker CellTrace™ violet-labeled cells (indicating proliferation cells) over the CD3+/7-ADD-cells (indicating total T cells) was calculated. The results indicate that cells treated with HCB301-3, HCB301-4, and HCB301-1 did not significantly change the cell proliferation as compared to that of the control molecules or combinations thereof.

As shown in FIG. 14B, IL-2 secretion in the MLR assays was determined using the Human IL-2 ELISA MAX Deluxe kit. The results showed that M7824 exhibited the highest IL-2 secretion level, followed by HCB301-1, HCB301-3, and HCB301-4. In particular, cells treated with HCB301-4 had a similar IL-2 secretion level as compared to that of IgG4-TGFβ trap and SIRPα_G4+PD1_G4+IgG4-TGFβ trap. Cells treated with SIRPα_G4+IgG4-TGFβ trap or PD1_G4+IgG4-TGFβ trap had low IL-2 secretion levels.

As shown in FIG. 14C, IFN-γ secretion in the MLR assays was determined using the Human IFNγ ELISA MAX Deluxe kit. The results showed that HCB301-1 exhibited the highest IFN-γ secretion level, followed by M7824, PD1_G4+IgG4-TGFβ trap, HCB301-3, and HCB301-4. Cells treated with PD1_G4, SIRPα_G4, SIRPα_G4+PD1_G4, SIRPα_G4+PD1_G4+IgG4-TGFβ trap, SIRPα_G4+PD1_G4, and SIRPα_G4+IgG4-TGFβ trap had similar and low IFN-γ secretion levels.

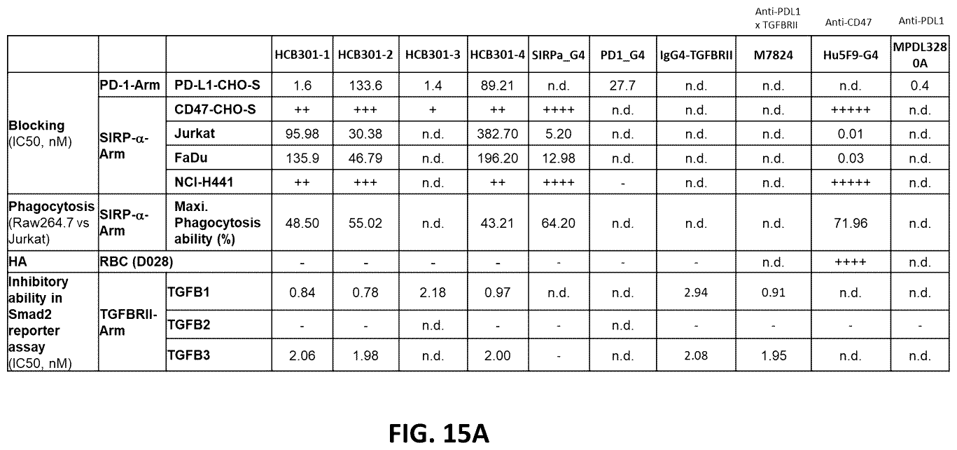

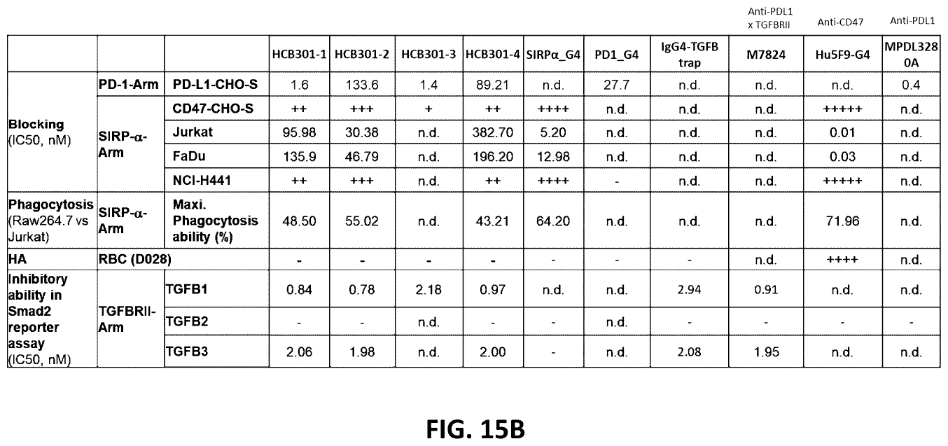

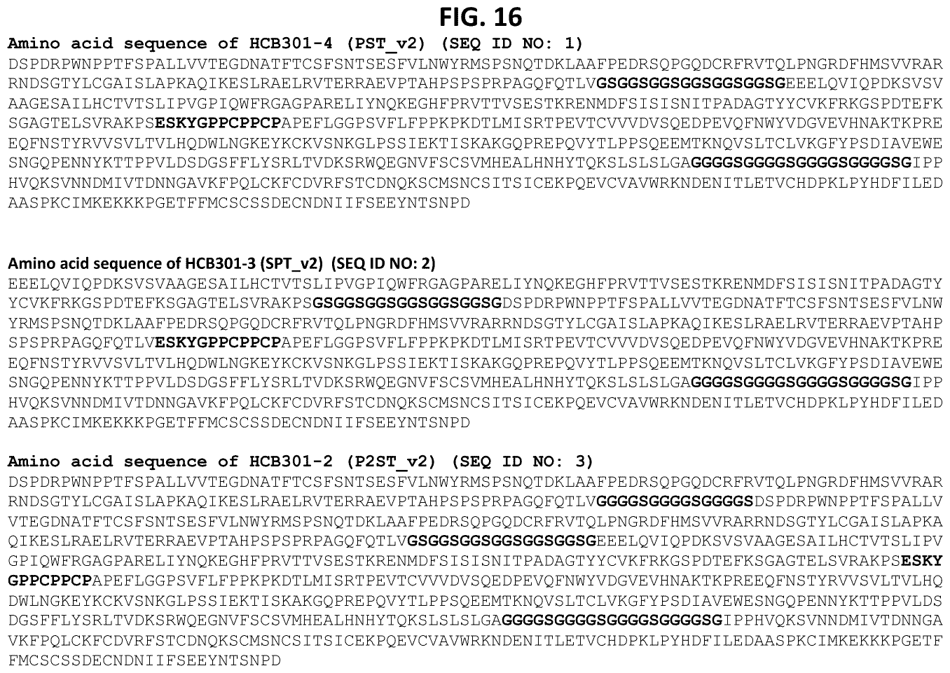

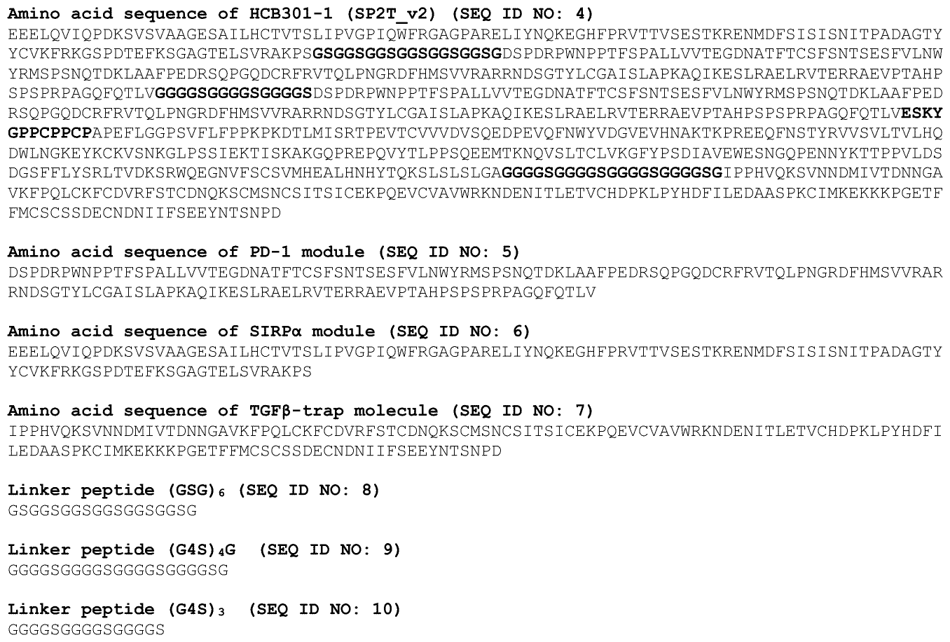

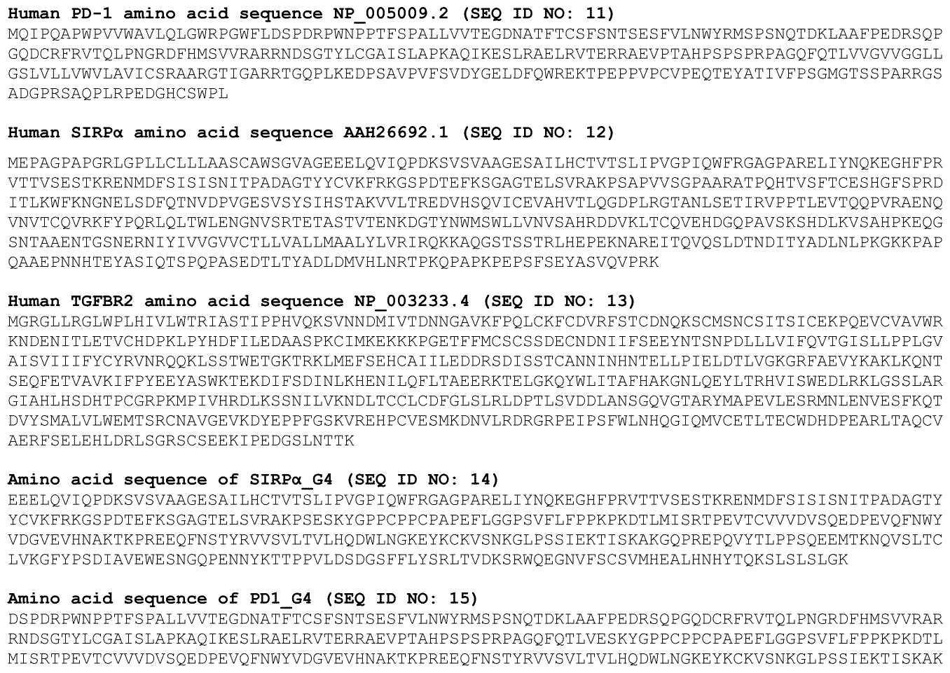

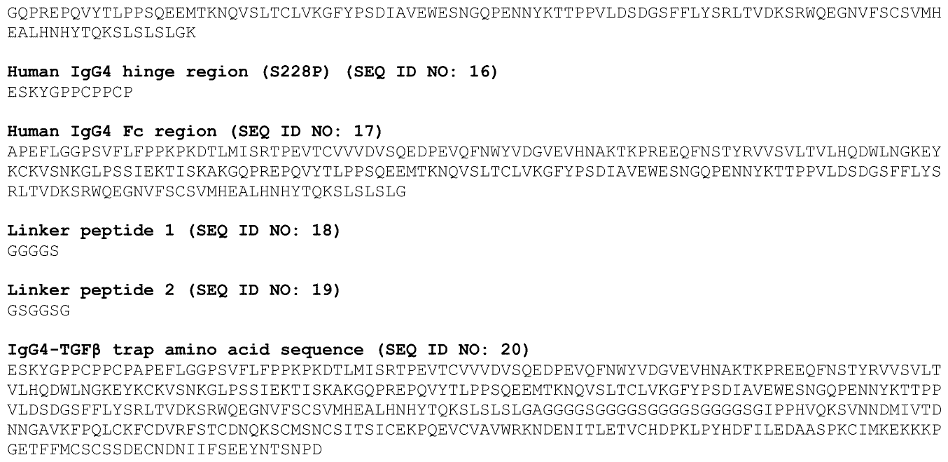

As a summary, FIGS. 15A-15B show the in vitro assay results as discussed above. HCB301-3 was selected for subsequent experiments, partially because of its relatively simple structure relative to HCB301-1 and HCB301-2, as well as its potent functions as shown the in vitro assays.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A protein complex, comprising:
(a) an Fc;
(b) a CD47-binding domain; and
(c) a PD-L1 (programmed death-ligand 1)-binding domain.

2. The protein complex of claim 1, further comprising a TGFβ (transforming growth factor beta)-binding domain.

3. The protein complex of claim 1, wherein the CD47-binding domain is or comprises a SIRPα extracellular domain, or wherein the CD47-binding domain is an anti-CD47 antibody or antigen-binding fragment thereof.

4. The protein complex of claim 1, wherein the PD-L1-binding domain is or comprises a PD-1 extracellular domain, or wherein the PD-L1-binding domain is an anti-PD-L1 antibody or antigen-binding fragment thereof.

5. The protein complex of claim 2, wherein the TGFβ-binding domain is or comprises a TGFBR2 extracellular domain, or wherein the TGFβ-binding domain is an anti-TGFβ antibody or antigen-binding fragment thereof.

6. The protein complex of claim 1, wherein the Fc is human IgG4 Fc or IgG1 Fc.

7. The protein complex of claim 2, wherein the PD-L1-binding domain is linked to the N-terminus of a CH2 domain in the Fc, wherein the CD47-binding domain is linked to the N-terminus of the PD-L1-binding domain, wherein the TGFβ-binding domain is linked to the C-terminus of a CH3 domain in the Fc.

8. The protein complex of claim 2, wherein the protein complex comprises two or more CD47-binding domains, two or more PD-L1-binding domains, or two or more TGFβ-binding domains.

9. A protein complex, comprising
(a) a first polypeptide comprising from N-terminus to C-terminus: a first PD-L1-binding domain, a first CD47-binding domain, a first Fc region, and a first TGFβ-binding domain; and
(b) a second polypeptide comprising from N-terminus to C-terminus: a second PD-L1-binding domain, a second CD47-binding domain, a second Fc region, and a second TGFβ-binding domain.

10. The protein complex of claim 9, wherein the first polypeptide further comprises a third PD-L1 binding domain, and the second polypeptide further comprises a fourth PD-L1-binding domain, wherein the third PD-L1-binding domain is linked to the N-terminus of the first PD-L1 binding domain, wherein the fourth PD-L1-binding domain is linked to the N-terminus of the second PD-L1-binding domain.

11. A protein complex, comprising
(a) a first polypeptide comprising from N-terminus to C-terminus: a first CD47-binding domain, a first PD-L1-binding domain, a first hinge region, a first Fc region, and a first TGFβ-binding domain; and
(b) a second polypeptide comprising from N-terminus to C-terminus: a second CD47-binding domain, a second PD-L1-binding domain, a second hinge region, a second Fc region, and a second TGFβ-binding domain.

12. The protein complex of claim 11, wherein the first polypeptide further comprises a third PD-L1 binding domain, and the second polypeptide further comprises a fourth PD-L1-binding domain, wherein the third PD-L1-binding domain is fused between the first PD-L1-binding domain and the first hinge region, wherein the fourth PD-L1-binding domain is fused between the second PD-L1-binding domain and the second hinge region.

13. A nucleic acid comprising a polynucleotide encoding the protein complex of claim 1.

14. A vector comprising the nucleic acid of claim 13.

15. A cell comprising the nucleic acid of claim 13.

16. A method of producing a protein complex, the method comprising
(a) culturing the cell of claim 15 under conditions sufficient for the cell to produce the protein complex; and
(b) collecting the protein complex produced by the cell.

17. A protein conjugate comprising the protein complex of claim 1, covalently bound to a therapeutic agent.

18. A method of treating a subject having cancer, the method comprising administering a therapeutically effective amount of a composition comprising the protein complex of claim 1, to the subject.

19. The method of claim 18, wherein the subject has a cancer cell expressing CD47 or PD-L1.

20. The method of claim 18, wherein the cancer is breast cancer, prostate cancer, non-small cell lung cancer, pancreatic cancer, diffuse large B-cell lymphoma, mesothelioma, lung cancer, ovarian cancer, colon cancer, pleural tumor, glioblastoma, esophageal cancer, gastric cancer, synovial sarcoma, thymic carcinoma, endometrial carcinoma, stomach cancer, cholangiocarcinoma, head and neck cancer, blood cancer, or a combination thereof.

21. The method of claim 18, wherein the cancer is a solid tumor.

22. A method of decreasing the rate of tumor growth or killing a tumor cell, the method comprising contacting a tumor cell with an effective amount of a composition comprising the protein complex of claim 1.

23. A pharmaceutical composition comprising the protein complex of claim 1, and a pharmaceutically acceptable carrier.
